(19) United States
(12) Patent Application Publication
(10) Pub. No.: US 2024/0016633 A1
(43) Pub. Date: Jan. 18, 2024

(54) DEVICES FOR ACCESSING AND TREATING VENOUS SINUSES AND METHODS OF USE

(71) Applicant: Route 92 Medical, Inc., San Mateo, CA (US)

(72) Inventors: Michi E. Garrison, San Mateo, CA (US); Craig Bonsignore, San Mateo, CA (US); Warren T. Kim, San Mateo, CA (US); Tony M. Chou, San Mateo, CA (US); John Miller, San Mateo, CA (US); Joey English, San Mateo, CA (US)

(21) Appl. No.: 18/350,533

(22) Filed: Jul. 11, 2023

Related U.S. Application Data
(60) Provisional application No. 63/497,352, filed on Apr. 20, 2023.
(60) Provisional application No. 63/388,529, filed on Jul. 12, 2022.

(51) Int. Cl. A61F 2/962; A61B 17/22; A61F 2/90

(52) U.S. Cl. CPC A61F 2/962; A61B 17/22; A61F 2/90; A61F 2250/0098; A61B 2017/22079; A61F 2250/0018

(57) ABSTRACT

Methods and a system for performing a medical procedure at a treatment site of an occlusion in a dural venous sinus of a brain of a patient including advancing a catheter and a flexible inner member retrograde to a target location distal to a sigmoid sinus relative to an access point of entry, positioning the catheter at the treatment site in the dural venous sinus, removing the inner member from the patient; and treating the occlusion through the catheter to restore outflow of blood from the brain through the dural venous sinus. Related devices, systems, and methods are provided.

### CROSS-REFERENCE TO RELATED APPLICATIONS

This application claims the benefit of priority under 35 U.S.C. § 119(e) to co-pending U.S. Provisional Patent Application Serial Nos. 63/388,529, filed Jul. 12, 2022, and 63/497,352, filed Apr. 20, 2023. The disclosures of the applications are incorporated by reference in their entireties.

### BACKGROUND

The present disclosure relates generally to medical methods, devices, and systems for accessing and treating intracranial or dural venous sinuses. Cerebral veins empty into large venous sinuses (also known as dural venous sinuses). The venous sinuses of the brain are situated within the subarachnoid space that facilitate outflow of blood from the cerebral veins of the brain to the jugular vein in the neck and back into the heart. There are seven paired dural sinuses including the transverse, cavernous, petrosal, sphenoparietal, sigmoid, and basilar sinuses. There are five unpaired dural sinuses including sagittal, straight, occipital, and intercavernous sinuses.

Blockages can occur in these sinuses to impair venous outflow from the brain. For example, cerebral venous sinus thrombosis (CVST) occurs when a blood clot forms in the brain's venous sinuses. Thrombosis within the venous sinuses builds pressure in the vessels leading to swelling and hemorrhage in the brain. CVST typically affects the superior sagittal, transverse, and cavernous sinuses. Risk factors for CVST include thrombophilia, acquired prothrombotic states, such as infections such as SARS-CoV-2 leading to COVID-19, otitis, sinusitis, and mastoiditis; acquired prothrombotic states, such as pregnancy, antiphospholipid syndrome, and the puerperium; chronic inflammatory conditions, such as Wegener granulomatosis and sarcoidoisis, as well as trauma such as head injury, dehydration, and neurosurgical procedures. Swelling and hemorrhage due to high pressure built in the venous sinuses of the brain can also be due to non-thrombotic pathologies. In another example, Intracranial venous sinus stenosis is thought to play a role in the development of idiopathic intracranial hypertension (IIH), which may lead to visual disability and blindness. Arachnoid granulations or fibrous septae can create intrinsic discrete obstructions called “intrinsic stenosis”.

Venous sinus pathologies like CYST and IIH are typically treated medically by controlling the pressure and inhibiting clotting, such as with anticoagulants or fibrinolytic agents like recombinant tissue plasminogen activator (rtPA), or anti-platelet therapy (intravenous unfractionated heparin or low-molecular-weight heparin) to prevent thrombus propagation and increase recanalization. These agents can be administered systemically or directly to the area of the thrombus. These treatments have many drawbacks including allergic reactions, embolism, stroke, reperfusion arrhythmias and others, the most serious risk being cerebral infarction with hemorrhagic transformation or intracerebral hemorrhage.

Surgical treatments of venous stenosis and CVST may also be performed to control and relieve pressure, for example, by deploying a stent or shunt to improve flow through the veins and lower pressure. Other surgical treatments, such as deploying a catheter to the dural sinuses for direct catheter thrombolysis or mechanical thrombectomy can also be performed. These surgical treatments require direct catheter access to the venous sinuses. The navigation of the venous system is particularly problematic and difficult. The dural venous sinuses are located within deep, tortuous anatomy in an anatomical location that has potential for life-threatening outcomes. The cerebral venous system is far more variable than the arterial system. The variation can be among individuals and also between the hemispheres of the same brain. For example, tributaries leading into the various dural venous sinuses can be found along a particular sinus in non-standard, unpredictable locations patient-to-patient. The venous sinuses are also variable due to the presence of trabeculae or septations that can arise from the lateral or medial aspect of a sinus wall and transect the space. The dural venous sinuses also include structures in the inner wall called chordae willisii, which are thought to be flow-improving structures. These intraluminal bands (trabeculae, bridges, synechiae, septations) vary in structure. The dural venous sinuses can also incorporate arachnoid granulations or villi, which are microscopic herniations of the arachnoid membrane that can invaginate through the walls of the venous sinuses. Each of these characteristics increase the difficulty in locating and navigating to a target site, in the dural sinuses, for example the site of an occlusion or other treatment. Together with the risk of perforating the dural venous sinus wall, this difficulty in access has generally limited the effectiveness and acceptance of surgical treatments of the venous sinuses. Release of embolic material is also a particularly risk in venous clots because the venous flow takes the embolic material back to the right side of the heart and then the lungs.

In addition, standard endovascular stent designs may not be optimal for the venous sinuses. Stents have been designed for treating arterial blockages in primarily circular cross-sectioned vessels. Similar (and in some cases the same) devices have also been used to treat venous disease in peripheral venous vasculature with some success. However, the venous sinus lumens are encased in layers of the dura mater which are generally non-compliant. The sinus lumens are not round and not compliant. Circular implants do not uniformly appose the wall of the sinuses and may therefore lead to thrombotic complications or re-blockages.

Other treatments exist which require endovascular access of the venous sinuses. For example, the venous sinus may be a target site to position an implant for the monitoring and/or treatment of brain activity, for example electrodes for neural mapping and/or neural modulation.

Venous sinus treatments can be made difficult due to the large size of the vessels in relation to the catheter being delivered. Dural venous sinuses occupy a significant volume within the cranial cavity. Unlike on the arterial side of the cerebral vasculature, the deeper more distal sites within the intracranial venous sinuses can be nearly as large as the veins leading out of the cranium. The superior sagittal sinus (SSS) is typically considered the largest of the dural venous sinuses and can have a lumen diameter of 7.3 mm-8.8 mm depending on measurement technique used, whereas the proximal transverse sinus can be 7.5 mm-9.4 mm in diameter, the mid sigmoid sinus can be about 7.3 mm-10 mm in diameter, the proximal sigmoid sinus can be 8.6 mm-10.5 mm in diameter (Boddu et al. PLOS One 2018; 13(6):e0196275). Thus, distal treatment sites such as the superior sagittal sinus may be as large as the veins traversed to reach it. Treating the superior sagittal sinus by aspiration embolectomy to remove thrombosis has not been an effective treatment due to conventional neurovascular catheters being small, prone to clogging, and unable to achieve significant improvement in flow while the risk of perforation is high.

Difficulties in access also occur due to the rigid environment of the dura mater in which the venous sinuses lie. Tracking access catheters or stent delivery or other treatment catheters in arteries and veins that are fixed in position is much more challenging than when the vessels are surrounded by relatively compliant tissue, such as musculature. Not only is catheter advancement more difficult, but the risk of vessel injury is greater. This is especially true for catheters that are larger in diameter and closer to the cross sectional dimensions of the lumen.

### SUMMARY

In an aspect, described is a method of performing a medical procedure at a treatment site in a dural venous sinus of a brain of a patient including positioning a system of devices into an advancement configuration. The system of devices includes a catheter having a catheter lumen, an inner diameter and a distal end; and an inner member sized and shaped to slide within the catheter lumen. The inner member defines a single lumen and has a distal portion having a first outer diameter that tapers distally to a second outer diameter that is smaller than the first outer diameter. The inner member transitions in flexibility from a proximal end of the inner member to a distal end of the inner member, the distal end of the inner member being more flexible than the distal end of the catheter. When positioned in the advancement configuration, the inner member extends coaxially through the catheter lumen until the distal portion of the inner member is positioned distal to the distal end of the catheter. The method further includes advancing the catheter and the flexible inner member retrograde to a target location distal to a sigmoid sinus relative to an access point of entry while the system of devices is positioned in the advancement configuration; positioning the catheter at the treatment site in the dural venous sinus, the treatment site comprising an occlusion; removing the inner member from the patient; and treating the occlusion through the catheter to restore outflow of blood from the brain through the dural venous sinus.

The step of treating can include removing thrombotic material in the dural venous sinus by applying aspiration to the catheter. The step of treating can include stenting the occlusion in the dural venous sinus with a stent delivered through the catheter. The inner member can further include a proximal segment having a hypotube. The inner member can further include an intermediate segment having an unreinforced polymer having a first durometer and the distal portion includes a polymer different from the unreinforced polymer of the intermediate segment. The polymer of the distal portion can have a second durometer less than the first durometer. The distal portion can have a tapered portion that tapers distally from the first outer diameter of between 0.048″ and 0.080″ to the second outer diameter of about 0.031″ up to about 0.048″ over a length between 0.5 cm and 4.0 cm. The inner member can have a length configured to extend from outside a patient's body, through a femoral artery, and beyond the sigmoid sinus. The inner member tapered portion can have a taper angle of a wall of the tapered portion relative to a center line of the tapered portion is between 0.9 to 1.6 degrees. The inner member tapered portion can have a flexibility, shape, and taper length configured to be atraumatically delivered to a venous sinus.

The first outer diameter of the inner member and the inner diameter of the catheter lumen can have a close fit configured to allow the catheter and the inner member to be advanced in the advancement configuration through tortuous, branched venous anatomy to reach the target location for treatment in the distal venous sinuses and aids in preventing the distal end of the catheter from catching on branches and tributaries. The second outer diameter can be about ½ of the first outer diameter, about 40% of the first outer diameter, or about 65% of the first outer diameter. The second outer diameter can be at a distal-most terminus of the inner member. The single lumen of the flexible elongate body can have an inner diameter of less than 0.024 inches.

The method can further include extending a guidewire within the single lumen so that a distal-most end of the guidewire is housed within the inner member proximal to a distal opening from the single lumen. The lengths of the catheter and the inner member can be configured to extend to the venous sinus and the flexibility of the catheter advancement device is sufficient to reach the venous sinus.

In an interrelated implementation, provided is a method for performing a medical procedure at a treatment site in a dural venous sinus of a brain of a patient including advancing a catheter and a flexible inner member retrograde to a target location distal to a sigmoid sinus relative to an access point of entry; positioning the catheter at the treatment site in the dural venous sinus; removing the inner member from the patient; and advancing a treatment device through the catheter to the treatment site. The treatment device can include an aspiration device, an implant delivery device, or a cerebral treatment device.

In an interrelated implementation, provided is a method of performing a medical procedure at a treatment site in a dural venous sinus of a brain of a patient including advancing a system of devices retrograde to a target location distal to a sigmoid sinus relative to an access point of entry. The system of devices includes a catheter having a catheter lumen, an inner diameter and a distal end; and an inner member sized and shaped to slide within the catheter lumen. The inner member has a distal portion having a first outer diameter that tapers distally to a second outer diameter that is smaller than the first outer diameter. When positioned in the advancement configuration, the inner member extends coaxially through the catheter lumen and the distal portion of the inner member is positioned distal to the distal end of the catheter. The method further includes positioning the catheter at the treatment site in the dural venous sinus. The treatment site can include an occlusion and the method can further include treating the occlusion with an expandable implant to restore outflow of blood from the brain through the dural venous sinus.

The expandable implant can be a stent deployable from a delivery system from a compressed configuration to a first expanded configuration having a first cross-sectional shape. The stent can be designed to be formed from the first expanded configuration having the first cross-sectional shape into a second expanded configuration having a second cross-sectional shape. The second cross-sectional shape can be different from the first cross-sectional shape. The first cross-sectional shape can be rounded and the second cross-sectional shape can be non-round. The non-round second cross-sectional shape can be a multi-sided shape having a plurality of rounded corners and a plurality of substantially flat sides. The non-round second cross-sectional shape can be oval, square, or triangular. The second cross-sectional shape can be a distortion of the first cross-sectional shape. The second cross-sectional shape can be larger in diameter than the first cross-sectional shape.

In an interrelated implementation, provided is an implantable device including a tubular member defining a lumen having a longitudinal axis from a proximal end to a distal end of the tubular member. The tubular member is configured to transition from an unexpanded configuration towards a fully expanded, deployed configuration. The unexpanded configuration has a first cross-sectional shape taken perpendicular to the longitudinal axis that is substantially round without sides and the deployed configuration has a second cross-sectional shape taken perpendicular to the longitudinal axis that is substantially non-round and multi-sided.

The first cross-sectional shape can be circular, oval, or elliptical. The second cross-sectional shape can be square or triangular. The second cross-sectional shape can have rounded corners and generally flat sides. The rounded corners can have a tighter bend radius upon expansion of the tubular member and can be formed by thinner, more malleable struts than struts forming the generally flat sides formed by thicker, less malleable struts. The tubular member can be further configured to transition from the unexpanded configuration to a transitional configuration before transitioning into the deployed configuration. The tubular member is configured to be reoriented relative to the anatomy when in the transitional configuration and before transitioning to the deployed configuration. The transitional configuration can have a transitional cross-sectional shape taken perpendicular to the longitudinal axis that is substantially round without sides. The transitional cross-sectional shape can be the same shape or a distorted version of the first cross-sectional shape and have a transitional diameter that is larger than a diameter across the first cross-sectional shape. The second cross-sectional shape can be configured to mate with luminal dimensions of a venous sinus.

In an interrelated implementation, provided is a system for implantation of a device in a venous sinus luminal space. The system includes a delivery system having an inner member; and an outer member. The system includes a tubular member formed by a plurality of struts that define a lumen having a longitudinal axis from a proximal end to a distal end of the tubular member. The tubular member is mounted on the inner member and constrained by the outer member during delivery. The tubular member is configured to transition from an unexpanded configuration towards a fully expanded, deployed configuration. The unexpanded configuration has a first cross-sectional shape taken perpendicular to the longitudinal axis that is substantially round without sides and the deployed configuration has a second cross-sectional shape taken perpendicular to the longitudinal axis that is substantially non-round and multi-sided. The system further includes an outer catheter configured to receive the tubular member constrained by the outer member in the unexpanded configuration for delivery.

The inner member can include a recessed area located a distance away from a distal-most end of the inner member. The recessed area can have an outer diameter that is less than an outer diameter of the inner member proximal to the recessed area. The outer member can be configured to be retracted and the tubular member can be self-expandable upon retraction of the outer tubular member towards the deployed configuration. The recessed area can include at least one radiopaque marker configured to identify a location of the tubular member within the recessed area. The inner member can include an atraumatic distal end region. The first cross-sectional shape can be circular, oval, or elliptical and the second cross-sectional shape can be square or triangular. The second cross-sectional shape can have rounded corners and generally flat sides. The rounded corners have a tighter bend radius upon expansion of the tubular member and are formed by thinner, more malleable struts than struts forming the generally flat sides formed by thicker, less malleable struts. The tubular member is configured to be reoriented relative to the anatomy when in the transitional configuration and before transitioning to the deployed configuration. The tubular member can be further configured to transition from the unexpanded configuration to a transitional configuration before transitioning into the deployed configuration. The transitional configuration can have a transitional cross-sectional shape taken perpendicular to the longitudinal axis that is substantially round without sides. The transitional cross-sectional shape can be the same shape or a distorted version of the first cross-sectional shape and have a transitional diameter that is larger than a diameter across the first cross-sectional shape. The second cross-sectional shape is configured to mate with luminal dimensions of a venous sinus.

In some variations, one or more of the following can optionally be included in any feasible combination in the above methods, apparatus, devices, and systems. More details are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1A shows a catheter system for accessing an occlusion site in a dural venous sinus;

FIG. 1B shows the catheter system of FIG. 1A assembled;

FIG. 1C is a detail view of a distal end region of a catheter advancement element taken along circle C-C of FIG. 1A;

FIG. 1D is a detail view of a distal end region of a catheter advancing element having a rescue guidewire parked proximal of the distal opening;

FIG. 2A shows a schematic of a conventional guidewire centered by a microcatheter and penetrating an occlusion within a dural venous sinus, such as a superior sagittal sinus;

FIG. 2B shows a schematic of a catheter advancement element positioned within a dural sinus and the tapered distal tip region deflecting upon reaching an occlusion;

FIG. 2C shows a schematic of a catheter advancement element positioned within a venous sinus, and the tapered distal tip positioned between a chordae and the sinus wall, rather than the main lumen of the sinus;

FIG. 3 is a schematic of an implementation of a test rig for assessing deflection of a tapered distal tip region upon reaching an occlusion within the venous sinuses;

FIGS. 4A-4D illustrate a method of advancement of a catheter system towards an occlusion in the superior sigmoid sinus;

FIGS. 5A-5C illustrate an alternative method of advancement of a catheter system advanced over a guidewire;

FIG. 6A illustrates the structure of the dural venous sinuses and surrounding anatomy;

FIG. 6B is a schematic showing a conventional stent having a substantially round cross-sectional shape positioned within the dural venous sinus;

FIG. 6C is a schematic of a venous sinus stent having a non-round cross-sectional shape positioned within and conforming to the dural venous sinus;

FIGS. 7A-7B are unrolled, flat views of an implementation of a venous sinus stent in an unexpanded and expanded configuration, respectively;

FIG. 7C illustrates a venous sinus stent in a first expanded configuration having a first cross-sectional shape;

FIG. 7D illustrates the venous sinus stent of FIG. 7C formed into a second expanded configuration having a second cross-sectional shape that is different from the first cross-sectional shape;

FIG. 8 is a partial cut-away view of the distal section of a venous sinus stent delivery system.

It should be appreciated that the drawings are for example only and are not meant to be to scale. It is to be understood that devices described herein may include features not necessarily depicted in each figure.

### DETAILED DESCRIPTION

There is a need for devices, systems, and methods to safely and quickly access and treat the dural venous sinuses. In particular, there is a need to deliver large-bore devices deep within the dural venous sinuses of the brain to restore blood outflow from the brain quickly and while minimizing the risk of perforation, either through delivery of a stent or aspiration of the blockage. Delivery of large bore access devices to the dural venous sinuses is also beneficial to the delivery of endovascular implants to target sites in the venous sinuses, for example the sagittal venous sinus.

There is also a need for venous stents and stent delivery systems that are optimized for implantation into the dural venous sinuses.

As mentioned above, surgical treatments of venous stenosis and CVST may be performed by deploying a stent or shunt to improve flow through the veins and lower pressure in the brain. Direct catheter thrombolysis, aspiration thrombectomy, and/or mechanical thrombectomy also can be performed. These surgical treatments require direct catheter access to the venous sinuses. The cerebral venous system can be divided into superficial and deep systems. For example, the superficial system including the superior sagittal sinus and the deep system includes the lateral sinus, straight sinus, sigmoid sinus and others. Regardless whether the sinus is deep or more superficial they each drain into the internal jugular veins. Catheters can navigate in retrograde fashion to access the dural venous sinuses past relatively tortuous anatomy. The jugular bulb is a dilation of the upper bulbous portion of the jugular vein. A first severe turn a catheter takes occurs between the jugular bulb and the sigmoid sinus. A second turn occurs between the sigmoid sinus and the transverse sinus. In addition to these tortuous pathways, the superficial sinuses are far more variable and non-standard than the arterial system. Tributaries leading into the various dural venous sinuses are located at non-standard sites along the sinuses and are relatively unpredictable patient-to-patient. The dural venous sinuses can incorporate trabeculae or septations or arachnoid granulations or chordae willisii that pose difficulties for conventional catheter systems to navigate efficiently and without too great of a risk of perforating the dural venous sinus wall. In the case of thrombosis in the dural venous sinuses, more than one attempt is typically made to completely remove the occlusion.

For thrombotic occlusions in the larger venous sinuses, such as the superior sagittal sinus, which can have a diameter of about 7.3 mm-8.8 mm or a total area of about 30.4 mm²-38.7 mm², removing the thrombotic occlusions within this space can require multiple passes of the catheter, sometimes as many as 30 passes, before the outflow is sufficiently achieved. Catheters tend to get clogged with thrombotic material, particularly where the catheter lumen size is smaller (e.g., less than about 0.070″ (1.778 mm)), and must be withdrawn, cleared, and re-advanced to attempt another clot removal. Each attempt is associated with potential procedural risk due to catheter system advancement through the tortuous and non-standard anatomy of the venous sinuses. Reducing the number of attempts by maximizing aspiration catheter lumen size (i.e., no less than 0.088″ (2.235 mm) and preferably as close to being size-matched to the sinus being treated) as well as reducing the time required to exchange devices for additional attempts are important factors in minimizing the overall time to perform a successful intervention.

Treating the superior sagittal sinus by aspiration embolectomy to remove thrombosis benefits from delivering a suitably large aspiration catheter (e.g., 0.088″ inner diameter or greater). Similarly, a stent deployed to treat a stenosis of a large venous sinus should be suitably large. Large stents though require a correspondingly large stent delivery system, which can cause issues navigating the tortuous anatomy of the intracranial venous system. The stent and stent size can vary depending on the sinus to be treated including carotid stents, such as WALLSTENT (Boston Scientific), PRECISE Stent (Cordis), ZILVER (Cook), and others that can be about 6 mm up to about 10 mm in diameter and are delivered through delivery catheters that have an inner diameter of about 0.070″ up to about 0.088″.

Large-bore access systems are also beneficial in delivering relatively large neurovascular implants and devices, such as stents and stent delivery systems, to the venous sinuses. Typically, larger catheters, particularly catheters carrying relatively large stents, can be limited in their ability to reach distal sites due to lack of navigability and also limited length. As mentioned above, the path to reach distal sites undergoes tight turns (e.g., internal jugular to sigmoid sinus) and incorporates non-standard anatomy (e.g. tributaries feeding the venous sinuses) hampered by the presence of irregular “intrinsic” stenosis caused by arachnoid granulations and septations forming parallel channels. Conventional catheter systems incorporating, for example, guidewires extending through microcatheters, tend to find unusual pathways through the micropassages creating delivery challenges and risks.

A further difficulty in navigating the venous sinuses with a total occlusion or stenosis is that it is typically performed “blind”. Determination of the location, size, and shape of a blockage for arterial occlusions is typically performed by fluoroscopic visualization after introduction of a radiopaque substance. Angiography is an industry standard for imaging vascular anatomy within the body. Angiography involves injection of contrast media and use of x-ray fluoroscopic imaging to visualize internal anatomy of the vasculature to evaluate blood flow, constrictions, or blockage, and to plan an appropriate treatment. Contrast media is introduced prior to or during imaging. The presence of the contrast media blocks or limits the ability of the x-rays to pass through. As a result, any region that temporarily contains the contrast media changes its appearance on the images. The x-ray angiography provides high resolution imaging showing the vasculature anatomical details. Contrast media is typically introduced intra-arterially (although it can also be introduced intravenously). Anatomic variability of the venous sinuses by cerebral angiogram makes diagnosis of an occlusion in the venous sinus insensitive. Unlike with an occlusion on the arterial side of the anatomy where the angiogram creates a roadmap to the occlusion, the sinuses fail to appear on an angiogram because the occlusion prevents contrast from reaching them. Intra-arterial contrast media is incapable of reaching the venous sinuses due to the blockage within the venous sinus. The vessels cannot be seen because the contrast never makes it into the sinus to replace the blood and absorb the x-rays. Access of an occlusion in a venous sinus is performed backwards or retrograde through the veins. Navigating the sinuses to the occlusion most typically is performed blind. It is possible to inject contrast directly into the venous side, but due to direction of flow the contrast may be visible only temporarily if at all. In some cases, one might inject through the catheter system or through the inner navigation catheter alone to get a direct venogram for orientation, for example, when the path is unclear based on where the catheter system is going or how the catheter system is behaving during navigation. Blind navigation of a conventional catheter system having a leading guidewire through the irregular venous sinus anatomy increases the risk of perforation as well as the risk of the catheter system being redirected into a tributary. Guidewires and microcatheters tend to get hung up on the tributaries, septations and granulations or are advanced through “false” lumens rather than the primary lumen, increasing the risk of perforation injuries and possibly leading to mis-deployments of stents in the false lumens.

The devices, systems, and methods described herein allow the user to safely navigate and optimally place treatment systems with respect to an occlusion of a venous sinus despite these visualization and navigational challenges. The devices, systems, and methods provide a safer way to find the larges lumens of the venous sinuses, particularly in the presence of intrinsic stenosis. The devices, systems, and methods enable safe and rapid positioning of large interventional devices, such as a large-bore aspiration catheters or stent delivery catheters to an occlusion in a dural venous sinus.

It has been found in performing the novel methods described herein that a novel structure is desirable to extend the range of applications of a conventional catheter to these novel treatment approaches. Provided herein are systems including a catheter advancement element having a tapered distal end region with a flexibility, shape, and taper length configured to be atraumatically delivered to a venous sinus. This is not achieved with conventional catheter systems as they may have improper flexibility, are formed of improper materials, or have improper shape and/or taper length resulting in conventional catheter systems getting misdirected or hung up or, if more force is applied, perforating the vessel. Unlike these conventional catheter systems, the catheter systems described herein includes a catheter advancement element capable of safely navigating variable anatomy of the dural venous sinuses and find the large lumen so that a corresponding large bore catheter (i.e., aspiration and/or stent delivery system) can be delivered to distal sites. The catheter systems described herein help locate and remove occlusions in venous sinuses in the novel manner of the methods provided herein. These and other features will be described in detail herein.

### Access System Components

The access catheter systems described herein can be used for treating various neurovascular pathologies of the dural venous sinuses, such as cerebral venous sinus thrombosis (CYST), idiopathic intracranial hypertension (IIH), intrinsic stenosis, and others. The systems described herein provide quick and simple single-operator access to distal target anatomy, in particular tortuous anatomy to the large venous sinuses in the brain at a single point of manipulation. The medical methods, devices and systems described herein allow for navigating large-bore catheters through complex, tortuous anatomy to perform rapid and safe treatment of occlusions. The medical methods, devices and systems described herein can also be used to deliver intracranial medical devices, with or without aspiration for the removal of and/or stenting of occlusions or stenosis in the treatment of these pathologies. The systems described herein can be particularly useful for the treatment of CVST or venous stenosis whether a user intends to perform aspiration alone as a frontline treatment for CVST or venous stenosis. The distal access catheter systems described herein can pass through tortuous loops past numerous arachnoid granulations, tributaries, and septations. The distal access catheter systems described herein can thereby create a safe conduit through the venous anatomy.

As used herein, “embolus” or “embolus material” or “embolic material” or “embolic region” refers to material within a zone of an occlusion site that is more dense or a relatively hard consistency that is preferably placed in contact with a distal end of an aspiration catheter to successfully perform aspiration embolectomy. The embolus may be a thrombus (a clot of blood) or other material that formed at a first blood vessel location (e.g., a coronary vessel), breaks loose, and travels through the circulation to a second blood vessel location. As used herein, “in situ thrombus” or “thrombus material” or “thrombotic material” or “thrombotic region” or “in situ clot material” or “clot material” refers to material within a zone of an occlusion site that accumulates in situ at the site of the embolus and is often less dense or relatively soft and fluid-like. As time passes, soft acute thrombus becomes firmer and more echogenic. Fibrin cross-links form and thrombus becomes more organized and adherent to the vessel wall. As used herein, “organized thrombus” refers to in situ thrombus material or clot material that accumulates at the site of embolus and has become more dense and less fluid-like than the acute in situ clot material. As used herein, “disorganized thrombus” refers to in situ thrombus material or clot material that accumulates at the site of embolus and is less dense and more fluid-like. As used herein, “an occlusion” or “an occlusion site” or “occlusive material” refers to the blockage that occurred as a result of an embolus lodging within a vessel and disrupting blood flow through the vessel or a stenosis within a vessel or sinus. The occlusion or occlusive material can include both thrombus and embolus as well as another non-thrombotic narrowing of the vessel.

The use of relative terms throughout the description may denote a relative position or direction. For example, “distal” may indicate a first direction away from a reference point, for example, distal with respect to the physician and the access point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the catheters and/or delivery systems to a specific configuration described in the various implementations.

As used herein, “vessel” refers to arteries and veins including the venous sinus. The venous sinuses include a branching sinus network that lies between layers of the dura mater, the outermost covering of the brain, and functions to collect oxygen-depleted blood from the brain. Unlike veins, these sinuses possess no inner layer of smooth muscle. Their lining is endothelium, a layer of cells like that which forms the surface of the innermost coat of the veins. The sinuses receive blood from the veins of the brain and connect directly or ultimately with the internal jugular vein.

While some implementations are described herein with specific regard to accessing a neurovascular anatomy for application of aspiration, the systems and methods described herein should not be limited to this and may also be applicable to other uses, such as the delivery of a stent deployment system. For example, the catheter systems described herein may be used to deliver working devices to the venous sinuses. Where the phrase “distal access catheter” or “aspiration catheter” is used herein that the catheter can be used for aspiration, the delivery of fluids to a treatment site or as a support catheter, or distal access providing a conduit that facilitates and guides the delivery or exchange of other devices, such as a guidewire or interventional devices, such as stents or stent retrievers.

The devices and systems described herein can be used in combination and in the alternative with the devices and systems described in U.S. Pat. No. 10,327,790, filed Aug. 3, 2012; U.S. Pat. No. 9,561,345, filed Dec. 19, 2014; U.S. Pat. No. 9,820,761, filed Feb. 4, 2016; U.S. Publication No. 2018/0193042, filed on Jan. 9, 2018; U.S. Publication No. 2018/0361114, filed on Jan. 19, 2018; U.S. Publication No. 2019/0351182, filed May 16, 2019; U.S. Pat. No. 11,400,255, filed Nov. 14, 2019; U.S. Publication No. 2020/0289136, filed Jun. 2, 2020; and U.S. Publication No. 2022/0111177, filed Oct. 8, 2021. The disclosures of each of these publications are incorporated by reference herein in their entireties.

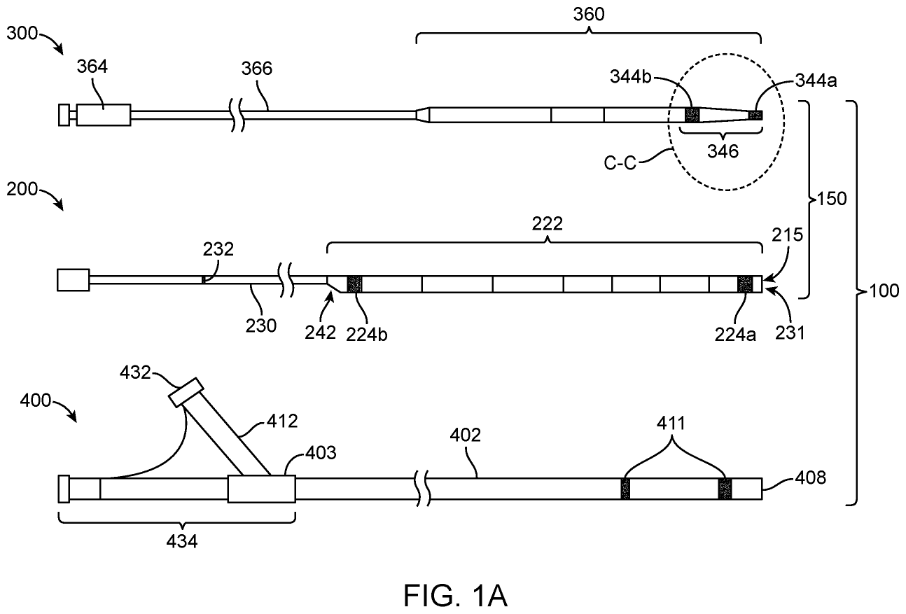

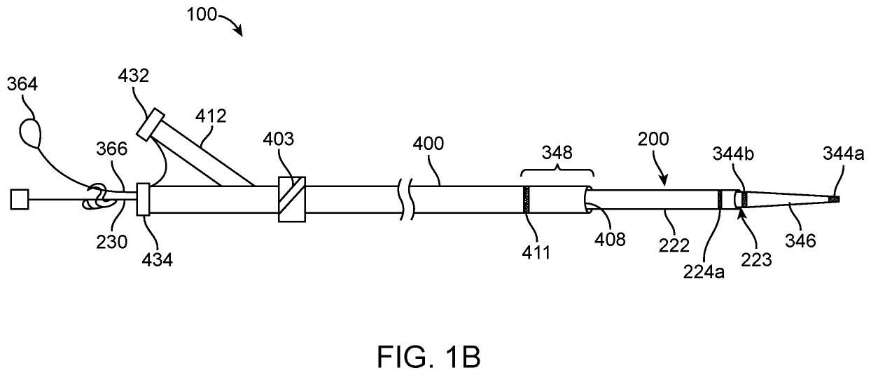

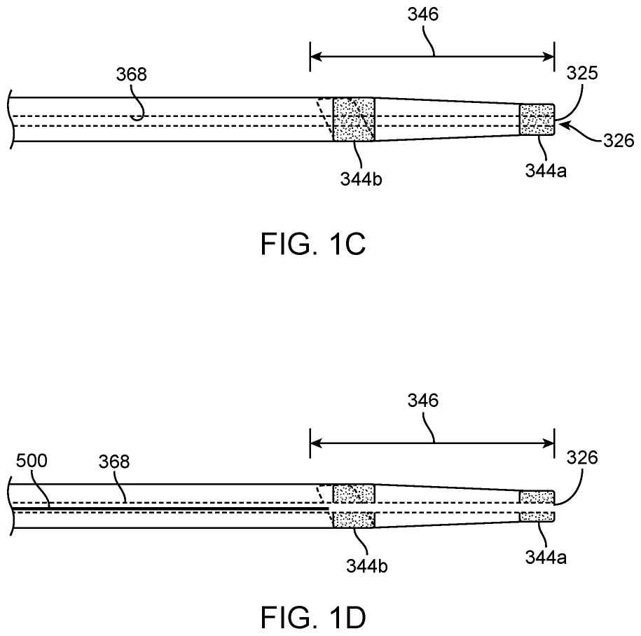

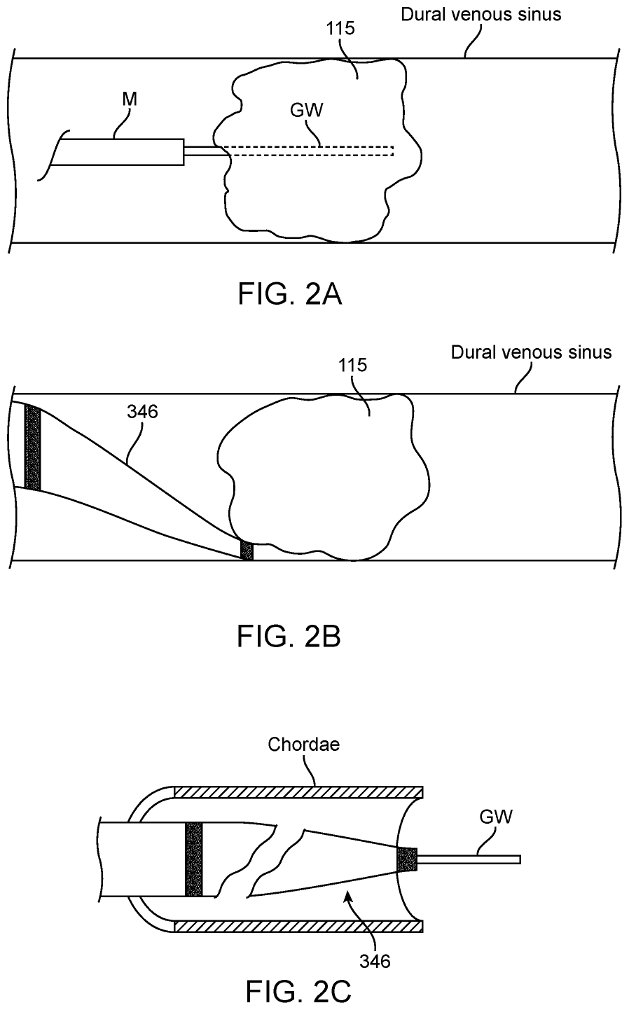

FIGS. 1A-1B illustrate an implementation of a distal access system 100 including devices for accessing a dural venous sinus to treat an occlusion, such as via aspiration embolectomy/thrombectomy and/or delivery of a stent. Where the system 100 is described herein as being useful for delivery of a stenting system, for example, it should be appreciated that the system 100 can also be used to deliver other systems, tools, or devices including electrodes for neural mapping and/or neural modulation. The system 100 can be used to apply aspiration and/or deliver contrast or other fluids through the system 100. Thus, terms like “access system” or “access catheter” might be used interchangeably with phrases, such as “aspiration system” or “aspiration catheter” or “delivery system” or “delivery catheter”. FIG. 1A is an exploded view of an implementation of a catheter system and FIG. 1B is an assembled view of the catheter system of FIG. 1A. FIG. 1C is a detailed view of the catheter advancement element of FIG. 1A taken along circle C-C. FIG. 1D is a detailed view of a catheter advancement element having a parked guidewire 500 in the lumen 368 having a distal-most end of the guidewire 500 housed within or positioned proximal to the distal opening 326 of the single lumen 368. The distal access system 100 is capable of providing quick and simple access to distal target anatomy, particularly the tortuous, non-standard anatomy of the dural venous sinus. The system 100 can be a single operator system such that each of the components and systems can be delivered and used together by one operator through a single point of manipulation requiring minimal hand movements. As will be described in more detail below, all manipulations can occur at or in close proximity to a single rotating hemostatic valve (RHV) or more than a single RHV co-located in the same device.

The system 100 can include one or more catheter systems 150, each having a catheter 200 and a catheter advancement element 300. The catheter system 150 is configured to be advanced through an access guide sheath 400. The catheter 200 is configured to be received through the guide sheath 400 and is designed to have exceptional deliverability. The catheter 200 can be a spined, distal access catheter co-axial with a lumen of the guide sheath 400 thereby providing a step-up in inner diameter within the conduit. The catheter 200 can also be a full length catheter. The catheter 200 can be delivered using a catheter advancement element 300 inserted through a lumen of the catheter 200. The flexibility and deliverability of the distal access catheter 200 allow the catheter 200 to take the shape of the tortuous anatomy and avoids exerting straightening forces creating new anatomy. The distal access catheter 200 is capable of this even in the presence of the catheter advancement element 300 extending through its lumen. The flexibility and deliverability of the catheter advancement element 300 is on par or better than the flexibility and deliverability of the distal luminal portion 222 of the distal access catheter 200. Both are configured to reach the superior sagittal sinus without straightening out the curves of the anatomy along the way, individually and assembled.

The system 100 can be a distal access system that can create a variable length from point of entry at the percutaneous arteriotomy (e.g. the femoral vein or other point of entry) to the target control point of the distal catheter. The distal access system 100 can include a long guide sheath or guide catheter placed through a shorter “introducer” sheath (e.g. 11-30 cm in length) at the groin. The long guide sheath can be positioned in the high jugular vein, the jugular bulb, or other high cervical vein to support interventions including embolectomy (sometimes referred to as “thrombectomy”) within the venous sinuses. For added support, the sheath can be advanced past the jugular bulb to a region of the transverse sinus. In some implementations, the sheath 400 can be used to advance a catheter and then the catheter used to bring support for the sheath 400 if a more distal site for sheath placement is desired. For example, to reach targets in the superior sagittal sinus, a catheter may be inserted through the long guide sheath to a first location and the guide sheath advanced over the catheter up to a region of the transverse sinus. These catheters, which can be large-bore aspiration catheters, can have a total working length of about 80 cm to about 120 cm, or more preferably about 90 cm up to about 115 cm when taken as a system as measured from the access point, typically the common femoral vein. The guide sheath 400 may have a length so that it hubs out at the incision at the access point and its distal end reaches a region of the transverse sinus. The single operator can use the systems described herein by inserting them through a single rotating hemostatic valve (RHV) 434 on the guide sheath 400 or more than one RHV co-located in the same device, such as a dual-headed RHV. Thus, what was once a two-person procedure can be a one-person procedure.

Still with respect to FIGS. 1A-1B, the access guide sheath 400 can have a body 402 through which a working lumen extends from a proximal hemostasis valve 434 coupled to a proximal end region 403 of the body 402 to a distal opening 408 of a distal end region. The working lumen is configured to receive the catheter 200 therethrough such that a distal end of the catheter 200 can extend beyond a distal end of the sheath 400 through the distal opening 408. The guide sheath 400 can be used to deliver the catheters described herein as well as any of a variety of working devices known in the art. For example, the working devices can be configured to provide thrombotic treatments and can include large-bore catheters for aspiration embolectomy (sometimes referred to as thrombectomy), advanced catheters, wires, balloons, stents, stent delivery systems, retrievable structures, such as coil-tipped retrievable stents “stent retriever”.

The sheath body 402 can extend from a proximal furcation or rotating hemostatic valve (RHV) 434 at a proximal end region 403 to a distal end 408 of the body 402. The proximal RHV 434 may include one or more lumens molded into a connector body to connect to the working lumen of the body 402 of the guide sheath 400. The working lumen can receive the catheter 200 and/or any of a variety of working devices for delivery to a target anatomy. The RHV 434 can be constructed of thick-walled polymer tubing or reinforced polymer tubing. The RHV 434 allows for the introduction of devices through the guide sheath 400 into the veins, while preventing or minimizing blood loss and preventing air introduction into the guide sheath 400. The RHV 434 can be integral to the guide sheath 400 or the guide sheath 400 can terminate on a proximal end in a female Luer adaptor to which a separate hemostasis valve component, such as a passive seal valve, a Tuohy-Borst valve or RHV may be attached. The RHV 434 can have an adjustable opening that is open large enough to allow removal of devices that have adherent clot on the distal end 408 without causing the clot to dislodge at the RHV 434 during removal. Alternately, the RHV 434 can be removable, such as when a device is being removed from the sheath 400 to prevent clot dislodgement at the RHV 434. The RHV 434 can be a dual RHV or a multi-head RHV.

The RHV 434 can form a Y-connector on the proximal end 403 of the sheath 400 such that the first port of the RHV 434 can be used for insertion of a working catheter into the working lumen of the sheath 400 and a second port into arm 412 can be used for another purpose. For example, a syringe or other device can be connected at arm 412 via a connector 432 to deliver a forward drip, a flush line for contrast agent or saline injections through the body 402 with or without a catheter toward the distal end 408 and into the target anatomy. Arm 412 can also connect to a vacuum source. The vacuum source can be an active source of aspiration, such as an aspiration pump, a regular or locking syringe, a hand-held aspirator, hospital suction, or the like, configured to draw suction through the working lumen. In an embodiment, the vacuum source is a locking syringe (for example a VacLok Syringe) attached to a flow controller. The user can pull the plunger on the syringe back into a locked position while the connection to the flow line is closed prior to an embolectomy step of the procedure. During the procedure when the distal-most end 215 of the catheter 200 is near or at the occlusion and the catheter advancement element 300 is removed from the lumen of the catheter 200, the user may open the connection to the aspiration syringe. This allows for a maximum communication of aspiration force being applied through the working lumen of the sheath 400 and any catheter extending through the sheath 400 that in turn is in communication with the vein at its distal end. A single user at the single, shared source can apply the aspiration in a rapid fashion. In another implementation, the arm 412 can be connected to a vacuum source that is a pump configured to apply a constant or variable aspiration pressure through the working lumen of the guide sheath 400. The single, shared source of aspiration is sufficient to draw aspiration through the entire system 100, even when multiple aspiration catheters 200 are nested within one another through the working lumen of the guide sheath 400. The working lumen can extend from the distal end 408 to a working proximal port of the proximal end region 403 of the sheath body 402.

Contrast agent can be injected through a second guide sheath 400 positioned into an artery to visualize the occlusion site by angiogram. For example, the arterial guide sheath 400 can be positioned so that at least a portion is positioned within the carotid artery. The contrast agent may be injected through the arterial guide sheath 400 once positioned in this location. A baseline angiogram can be obtained, for example in the anterior/posterior (AP) and/or lateral views, prior to device insertion to assess occlusion location by injection of contrast media through the arterial guide sheath 400 with fluoroscopic visualization. Fluoroscopic visualization may continue as the catheter system is advanced and subsequent angiograms can be captured periodically and particularly after every attempt to retrieve the embolus to assess reperfusion. The baseline angiogram image can be superimposed, such as with digital subtraction angiography, so that the occlusion site is visible while the catheter system is advanced. As discussed above, contrast injected from the arterial side may not reach the venous side due to the presence of the occlusion. In some implementations, the sheath 400 positioned within the venous sinuses may be used to deliver contrast and perform a venogram. A venogram is an x-ray test that involves injecting contrast material into a vein to show how blood flows through the veins. This allows a physician to determine the condition of the veins and locate thrombus. Utility of contrast injection on the venous side, however, is limited resulting in navigation to an occluded sinus being performed essentially blind.

Once the catheter system 150 is advanced into position (the advancement and positioning will be described in more detail below), the catheter advancement element 300 can be withdrawn and removed from the system. In some implementations, the catheter 200 can be used as a support catheter to deliver a stent to the occlusion site within the venous sinus as will be described elsewhere herein. The catheter 200 can be used to perform aspiration thrombectomy at the occlusion site within the venous sinus. For example, a vacuum source, such as a pump, may be connected to the venous sheath 400 and activated to direct aspiration to the distal end of the catheter 200 located within the venous sinus. The aspiration may be applied for a period of time (e.g., between about 30 seconds up to about 3 minutes, preferably about 2 minutes) to allow for capture and engulfment of the embolus in the catheter 200. The flow rate of aspiration may vary and in one example can be between about 25 inches Hg (inHg) (12.279 psi) up to about 28 inHg (13.752 psi). In some implementations, the pump is allowed to run to build up a vacuum outside of the patient over a first period prior to applying the vacuum to the venous sinus, for example, by turning a flow control switch to an “on” position. In other implementations, the pump is turned on at a particular flow rate and is applied to the venous sinus immediately allowing for the build-up of vacuum through the system. After applying aspiration to the catheter for a period of time, the catheter 200 can be slowly withdrawn. The catheter 200 can be re-advanced and withdrawn until sufficient amounts of thrombotic material from one or more venous sinuses is removed to establish outflow from the brain. Once free flow is achieved, observable by continuous collection of fluid within a receptacle, the aspiration source can be disconnected from the venous sheath 400 and a confirmatory angiogram performed by injecting contrast agent through the working lumen of the arterial sheath 400.

The vacuum source can increase in aspiration level when the flow rate is slow and decrease when the flow rate is increased. In this manner, the force is greatest when the catheter is clogged or partially clogged, but decreases to a minimal level when there is free flow to ensure protection from emboli but limit the volume of aspirated blood. In this manner, the system can optimize the embolus aspiration while limiting the amount of blood aspirated. Alternately, the vacuum source can include a vacuum gauge. When the flow in the catheter 200 is blocked or restricted, the pump can create a higher level of vacuum. In this example, the aspiration force may be configured to rise when higher vacuum is detected. Alternatively, the vacuum gauge may be incorporated into the RHV or the Luer or proximal end of the guide sheath 400.

In an implementation, the guide sheath 400 includes one or more radiopaque markers 411. The radiopaque markers 411 can be disposed near the distal end 408. For example, a pair of radiopaque bands may be provided. The radiopaque markers 411 or markers of any of the system components can be swaged, painted, embedded, or otherwise disposed in or on the body. In some implementations, the radiopaque markers include a barium polymer, tungsten polymer blend, tungsten-filled or platinum-filled marker that maintains flexibility of the devices and improves transition along the length of the component and its resistance to kinking. In some implementations, the radiopaque markers are a tungsten-loaded PEBAX or polyurethane that is heat welded to the component.

The guide sheath markers 411 are shown in the figures as rings around a circumference of one or more regions of the body 402. However, the markers 411 can have other shapes or create a variety of patterns that provide orientation to an operator regarding the position of the distal opening 408 within the vessel. Accordingly, an operator may visualize a location of the distal opening 408 under fluoroscopy to confirm that the distal opening 408 is directed toward a target anatomy where a catheter 200 is to be delivered. For example, radiopaque marker(s) 411 allow an operator to rotate the body 402 of the guide sheath 400 at an anatomical access point, e.g., a groin of a patient, such that the distal opening provides access to a vein by subsequent working device(s), e.g., catheters and wires advanced to the venous sinus. In some implementations, the radiopaque marker(s) 411 include platinum, gold, tantalum, tungsten or any other substance visible under an x-ray fluoroscope. Any of the various components of the systems described herein can incorporate radiopaque markers.

Still with respect to FIGS. 1A-1B, the catheter 200 can include a relatively flexible, distal luminal portion 222 coupled to a stiffer, kink-resistant proximal extension or proximal control element 230. The term “control element” as used herein can refer to a proximal region configured for a user to cause pushing movement in a distal direction as well as pulling movement in a proximal direction. The control elements described herein may also be referred to as spines, tethers, push wires, push tubes, or other elements having any of a variety of configurations. The proximal control element 230 can be a hollow or tubular element. The proximal control element 230 can also be solid and have no inner lumen, such as a solid rod, ribbon or other solid wire type element. Generally, the proximal control elements described herein are configured to move its respective component (to which it may be attached or integral) in a bidirectional manner through a lumen.

A single, inner lumen 223 extends through the luminal portion 222 between a proximal end and a distal end of the luminal portion 222 (the lumen 223 is visible in FIG. 1B). In some implementations, a proximal opening 242 into the lumen 223 can be located near where the proximal control element 230 couples with the distal luminal portion 222. In other implementations, the proximal opening 242 into the lumen 223 is at a proximal end region of the catheter 200. A distal opening 231 from the lumen 223 can be located near or at the distal-most end 215 of the luminal portion 222. The inner lumen 223 of the catheter 200 can have a first inner diameter and the working lumen of the guide sheath 400 can have a second, larger inner diameter. Upon insertion of the catheter 200 through the working lumen of the sheath 400, the lumen 223 of the catheter 200 can be configured to be fluidly connected and contiguous with the working lumen of the sheath 400 such that fluid flow into and/or out of the system 100 is possible, such as by applying suction from a vacuum source coupled to the system 100 at a proximal end. The combination of sheath 400 and catheter 200 can be continuously in communication with the bloodstream during aspiration at the proximal end with advancement and withdrawal of catheter 200.

The distal luminal portion 222 of the catheter 200 can have a plurality of radiopaque markings 224. A first radiopaque marker 224a can be located near the distal-most end 215 to aid in navigation and proper positioning of the distal-most end 215 under fluoroscopy. Additionally, a proximal region of the catheter 200 may have one or more proximal radiopaque markers 224b so that the overlap region 348 can be visualized as the relationship between a radiopaque marker 411 on the guide sheath 400 and the radiopaque marker 224b on the catheter 200. The proximal region of the catheter 200 may also have one or more radiopaque markings providing visualization, for example, near the proximal opening 242 into the single lumen 223 of the catheter 200 as will be described in more detail below. In an implementation, the two radiopaque markers (marker 224a near the distal-most end 215 and a more proximal marker 224b) are distinct to minimize confusion of the fluoroscopic image, for example the catheter proximal marker 224b may be a single band and the marker 411 on the guide sheath 400 may be a double band and any markers on a working device delivered through the distal access system can have another type of band or mark. The radiopaque markers 224 of the distal luminal portion 222, particularly those near the distal end region navigating extremely tortuous anatomy, can be relatively flexible such that they do not affect the overall flexibility of the distal luminal portion 222 near the distal end region. The radiopaque markers 224 can be tungsten-loaded or platinum-loaded markers that are relatively flexible compared to other types of radiopaque markers used in devices where flexibility is not paramount. In some implementations, the radiopaque marker can be a band of tungsten-loaded PEBAX having a durometer of Shore 35D.

The proximal control element 230 can include one or more markers 232 to indicate the overlap between the distal luminal portion 222 of the catheter 200 and the sheath body 402 as well as the overlap between the distal luminal portion 222 of the catheter 200 and other interventional devices that may extend through the distal luminal portion 222. At least a first marker can be an RHV proximity marker positioned so that when the marker is aligned with the sheath proximal hemostasis valve 434 during insertion of the catheter 200 through the guide sheath 400, the catheter 200 is positioned at the distal-most position with the minimal overlap length needed to create the seal between the catheter 200 and the working lumen. At least a second marker 232 can be a Fluoro-saver marker that can be positioned on the control element 230 and located a distance away from the distal-most end 215 of the distal luminal portion 222. In some implementations, a marker 232 can be positioned about 100 cm away from the distal-most end 215 of the distal luminal portion 222. The markers 232 can be positioned on the catheter so that one or more markers are visible to an operator outside the patient (and outside the guide sheath 400) during use. One or more markers can also be visible to an operator inside the patient (and inside the guide sheath 400 or beyond a distal end of the guide sheath 400) during use such that they are visualized under fluoroscopy.

Although the catheter advancement element 300 is described herein in reference to catheter 200, it can be used to advance any of a variety catheters and it is not intended to be limiting to its use. For example, the catheter advancement element 300 can be used to deliver standard, full-length catheters, such as a 5MAX Reperfusion Catheter (Penumbra, Inc. Alameda, CA), BENCHMARK 071 (Penumbra), REACT aspiration catheter (Medtronic), IKARI catheter (Asahi), or Sophia Plus aspiration catheter (Terumo). In preferred embodiments, the catheter advancement element 300 is size-matched to a large bore catheter 200 that is at least 0.088″ inner diameter up to about 0.120″ up to about 0.160″.

Still with respect to FIGS. 1A-1B and also FIG. 1C, the catheter advancement element 300 can include a non-expandable, flexible elongate body 360 and a proximal portion 366 extending proximally from the elongate body 360. The catheter advancement element 300 and the catheter 200 described herein may be configured for rapid exchange or over-the-wire methods. For example, the flexible elongate body 360 can be a tubular portion extending the entire length of the catheter advancement element 300 and can have a proximal opening from the lumen 368 of the flexible elongate body 360 that is configured to extend outside the patient's body during use. Alternatively, the tubular portion can have a proximal opening positioned such that the proximal opening remains inside the patient's body during use. The proximal portion 366 can be a proximal element coupled to a distal tubular portion 360 and extending proximally therefrom. A proximal opening from the tubular portion 360 can be positioned near where the proximal element 366 couples to the tubular portion 360. Alternatively, the proximal portion 366 can be a proximal extension of the tubular portion 360 having a length that extends to a proximal opening near a proximal terminus of the catheter advancement element 300 (i.e., outside a patient's body). A luer 364 can be coupled to the proximal portion 366 at the proximal end region so that tools, such as a guidewire can be advanced through the lumen 368 of the catheter advancement element 300. A syringe or other component can be coupled to the luer 364 in order to draw a vacuum and/or inject fluids through the lumen 368. The syringe coupled to the luer 364 can also be used to close off the lumen of the catheter advancement element 300 to maximize the piston effect described elsewhere herein.

The configuration of the proximal portion 366 can vary. In some implementations, the proximal portion 366 is simply a proximal extension of the flexible elongate body 360 that does not change significantly in structure but changes significantly in flexibility. For example, the proximal portion 366 transitions from the very flexible distal regions of the catheter advancement element 300 towards less flexible proximal regions of the catheter advancement element 300. The proximal portion 366 provides a relatively stiff proximal end suitable for manipulating and torqueing the more distal regions of the catheter advancement element 300. In other implementations, the proximal portion 366 is a metal reinforced segment. The metal reinforced segment can be positioned a distance away from the distal end of the elongate body. For example, the metal reinforced segment can terminate or be about 50 cm from the distal end. The metal reinforced segment can have an inner diameter of about 0.021″ and an outer diameter of about 0.027″. The metal reinforced segment can be a spine. The metal reinforced segment can be a hypotube. In other implementations, the proximal portion 366 is a hypotube coupled to the elongate body 360. The hypotube may be exposed or may be coated by a polymer. In still further implementations, the proximal portion 366 may be a tubular polymer portion reinforced by a coiled ribbon or braid. The proximal portion 366 can have the same outer diameter as the flexible elongate body or can have a smaller outer diameter as the flexible elongate body.

The proximal portion 366 need not include a lumen. For example, the proximal portion 366 can be a solid rod, ribbon, or wire have no lumen extending through it that couples to the tubular elongate body 360. Where the proximal portion 366 is described herein as having a lumen, it should be appreciated that the proximal portion 366 can also be solid and have no lumen. The proximal portion 366 is generally less flexible than the elongate body 360 and can transition to be even more stiff towards the proximal-most end of the proximal portion 366. Thus, the catheter advancement element 300 can have an extremely soft and flexible distal end region 346 that transitions proximally to a stiff proximal portion 366 well suited for pushing the distal elongate body 360. The distal elongate body 360 of the catheter advancement element 300 is substantially non-reinforced, fully polymeric body. In other words, the distal elongate body 360 can include no reinforcement element (i.e., metal coil or braid, stiff polymer, etc.) such that the stiff proximal portion 366 can be used for pushing the distal elongate body 360, but may not necessarily be useful in torqueing the distal elongate body 360. In other implementations, the distal elongate body 360 of the catheter advancement element 300 can be reinforced such that the proximal portion 366 can be used for pushing and also torqueing the distal elongate body 360. In some implementations, the catheter advancement element 300 can be inserted and advanced through and beyond the catheter 200 by pushing on the proximal portion 366. Alternately, the catheter system can be advanced as a whole by pushing on the combination of the catheter 200 and the catheter advancement element 300. For example, the proximal control element 230 of catheter 200 and the elongate body 360 of the catheter advancement element 300 can be held and advanced together by the user. In another example, the control element 230 of the catheter 200 and the elongate body 360 of the catheter advancement element 300 can be held together by a separate mechanical clamp as described below, and then advanced together.

The elongate body 360 can be received within and extended through the internal lumen 223 of the distal luminal portion 222 of the catheter 200 (see FIG. 1B). The elongate body 360 or tubular portion can have an outer diameter. The outer diameter of the tubular portion can have at least one snug point. The at least one snug point provides a close fit between the elongate body 360 and the distal luminal portion 222 that minimizes a distal lip or edge at the distal end of the catheter 200, but that still allows for movement relative to one another so as to allow a user to achieve a desired extension or withdrawal of the catheter advancement element 300 relative to the catheter 200 or the catheter 200 relative to the catheter advancement element 300. The snug point allows for movement between the catheters upon application of a relatively small, but non-zero force that does not overly tension or stretch the components, so as to avoid damage to the devices and any negative impact on usability within a patient. A difference between the inner diameter of the catheter 200 and the outer diameter of the tubular portion at the snug point can be no more than about 0.015″ (0.381 mm), or can be no more than about (0.254 mm), for example, from about 0.003″ (0.0762 mm) up to about 0.012″ (0.3048 mm), preferably about 0.005″ (0.127 mm) to about 0.010″ (0.254 mm), and more preferably about 0.007″ (0.1778 mm) to about 0.009″ (0.2286 mm). The size difference between the catheter advancement element 300 and the catheter 200 (i.e., the annular gap at the snug point) can be as small as 0.000″-0.001″ depending on the material(s) of the components and/or the length of the region. For example, if the materials of the inner and outer catheters are soft, they may also feel “stickier” when extending one relative to the other so that a larger annular gap is preferable than if another material is used. As another example, hydrophilic coatings on a component, even if that component is made of a soft material that normally has a “sticky” feel, can allow for a smaller annular gap without negatively impacting the telescoping function of the components. The overall length of the snug region can also dictate the preferred size difference to achieve a desired feel. A longer length of the snug region might have a larger annular gap and provide a snug feel.

As will be described in more detail below, the catheter advancement element 300 can also include a distal end region 346 located distal to the at least one snug point of the tubular portion. The distal end region 346 can have a length and taper along at least a portion of the length. The distal end region 346 of the catheter advancement element 300 can be extended beyond the distal end of the catheter 200 as shown in FIG. 1B. The proximal portion 366 of the catheter advancement element 300 or proximal extension is coupled to a proximal end region of the elongate body 360 and extends proximally therefrom. The proximal portion 366 can be less flexible than the elongate body 360 and configured for bi-directional movement of the elongate body 360 of the catheter advancement element 300 within the luminal portion 222 of the catheter 200, as well as for movement of the catheter system 100 as a whole. The elongate body 360 can be inserted in a coaxial fashion through the internal lumen 223 of the luminal portion 222. The outer diameter of at least a region of the elongate body 360 can be sized to substantially fill at least a portion of the internal lumen 223 of the luminal portion 222.

The overall length of the catheter advancement element 300 (e.g. between the proximal end through to the distal-most tip) can vary, but generally is long enough to extend through the support catheter 200 plus at least a distance beyond the distal end of the support catheter 200 while at least a length of the proximal portion 366 remains outside the proximal end of the guide sheath 400 and outside the body of the patient. In some implementations, the overall length of the catheter advancement element 300 is about 145 to about 150 cm and has a working length of about 140 cm to about 145 cm from a proximal tab or hub to the distal-most end 325. The elongate body 360 can have a length that is at least as long as the luminal portion 222 of the catheter 200 although the elongate body 360 can be shorter than the luminal portion 222 so long as at least a minimum length remains inside the luminal portion 222 when a distal portion of the elongate body 360 is extended distal to the distal end of the luminal portion 222 to form a snug point or snug region with the catheter. In some implementations, this minimum length of the elongate body 360 that remains inside the luminal portion 222 when the distal end region 346 is positioned at its optimal advancement configuration is at least about 5 cm, at least about 6 cm, at least about 7 cm, at least about 8 cm, at least about 9 cm, at least about 10 cm, at least about 11 cm, or at least about 12 cm up to about 50 cm. In some implementations, the shaft length of the distal luminal portion 222 can be about 35 cm up to about 75 cm and shorter than a working length of the guide sheath and the insert length of the elongate body 360 can be at least about 45 cm, 46 cm, 47 cm, 48 cm, 48.5 cm, 49 cm, 49.5 cm up to about 85 cm.

The length of the elongate body 360 can allow for the distal end of the elongate body 360 to reach venous sinus targets or occlusions within, for example, the jugular bulb, transverse sinus, cavernous sinus, petrosal sinus, sphenoparietal sinus, sigmoid sinus, basilar sinus, sagittal sinus, straight sinus, occipital sinus, intercavernous sinus, and others. The distal end region of the elongate body 360 can reach these distal target locations while the proximal end region of the elongate body 360 remains proximal to or below the level of severe turns along the path of insertion. For example, the entry location of the catheter system can be in the femoral vein and the target occlusion location can be distal to the jugular bulb, such as within the sigmoid sinus, transverse sinus, or superior sagittal sinus. The proximal end region of the elongate body 360 where it transitions to the proximal portion 366 can remain within a vessel that is proximal to severely tortuous anatomy, such as the within the internal jugular vein. This avoids inserting the stiffer proximal portion 366, or the material transition between the stiffer proximal portion 366 and the elongate body 360, from taking the turn of the sigmoid sinus, which can be very severe. The lengths described herein for the distal luminal portion 222 also can apply to the elongate body 360 of the catheter advancement element. The length of the catheter advancement element can be sized to extend from outside a patient's body, through a femoral artery and beyond the sigmoid sinus.

The proximal portion 366 can have a length that varies as well. In some implementations, the proximal portion 366 is about 90 cm up to about 95 cm. The distal portion extending distal to the distal end of the luminal portion 222 can include distal end region 346 that protrudes a length beyond the distal end of the luminal portion 222 during use of the catheter advancement element 300. The distal end region 346 of the elongate body 360 that is configured to protrude distally from the distal end of the luminal portion 222 during advancement of the catheter 200 through the tortuous venous sinuses having numerous non-standard branches and tributaries. Meaning, the location of the tributaries feeding into the larger venous sinuses can vary widely from patient-to-patient and are somewhat unpredictable for a surgeon to know, particularly where an angiogram provides little to no navigation guidance. The proximal portion 366 coupled to and extending proximally from the elongate body 360 can align generally side-by-side with the proximal control element 230 of the catheter 200. The arrangement between the elongate body 360 and the luminal portion 222 can be maintained during advancement of the catheter 200 through the tortuous, branched anatomy to reach the target location for treatment in the distal venous sinuses and aids in preventing the distal end of the catheter 200 from catching on tributaries, septations, and granulations within the sinuses, as will be described in more detail below.

In some implementations, the elongate body 360 can have a region of relatively uniform outer diameter extending along at least a portion of its length and the distal end region 346 tapers down from the uniform outer diameter. The outer diameter of the elongate body 360 also can taper or step down in outer diameter proximally, for example near where the elongate body 360 couples or transitions to the proximal portion 366. The outer diameter of the elongate body 360 need not change in outer diameter near where the elongate body 360 couples to the proximal portion 366. In some implementations, the region of relatively uniform outer diameter can extend along a majority of the working length of the catheter advancement element 300 including the proximal portion 366. This first region of uniform outer diameter can transition to a second region of uniform outer diameter located distal to the first region. The transition can incorporate a smooth taper or step change in outer diameter between the two regions. The second region of uniform outer diameter having the larger size and located distal to the first region can be useful in filling a lumen of a larger bore catheter without the entire working length of the elongate body needing to have this larger size. In this embodiment, the elongate body 360 can have a distal taper changing in diameter from the second uniform diameter region towards the distal opening and a proximal taper changing in diameter from the second uniform diameter region towards the first region of uniform outer diameter.

Depending upon the inner diameter of the catheter 200, the difference between the inner diameter of catheter 200 and the outer diameter of the elongate body 360 along at least a portion of its length, such as at least 10 cm of its length, preferably at least 15 cm of its length can be no more than about 0.015″ (0.381 mm), such as within a range of about 0.003″-0.015″ (0.0762 mm-0.381 mm) or between 0.006″-0.010″ (0.1524 mm-0.254 mm). Thus, the clearance between the catheter 200 and the elongate body 360 can result in a space on opposite sides that is no more than about 0.008″ (0.2032 mm), or can be no more than about 0.005″ (0.127 mm), for example, from about 0.001″ up to about 0.006″ (0.0254 mm-0.1524 mm), preferably about 0.002″ to about 0.005″ (0.0508 mm-0.127 mm), and more preferably about 0.003″ to about 0.005″ (0.0762 mm-0.0508 mm).

The elongate body 360 can have an overall shape profile from proximal end to distal end that transitions from a first outer diameter having a first length to a tapering outer diameter having a second length. The first length of this first outer diameter region (i.e. the snug-fitting region between the distal luminal portion 222 and the elongate body 360) can be at least about 5 cm, or 10 cm, up to about 50 cm. In other implementations, the snug-fitting region can extend from the proximal tab or luer 364 substantially to the tapered distal end region 346 which depending on the length of the catheter advancement element 300, can be up to about 170 cm. The first outer diameter of the elongate body 360 and the inner diameter of the catheter lumen can have a close fit configured to allow the catheter and the elongate body to be advanced together while in an advancement configuration (e.g., the elongate body 360 extending through the outer catheter so the distal tapered region extends a distance outside the distal end of the outer catheter) through tortuous, branched venous anatomy to reach the target location for treatment in the distal venous sinuses and aids in preventing the distal end of the catheter from catching on branches and tributaries.

The length of the tapering outer diameter of the distal end region 346 can be about 0.5 cm to about 5 cm, about 1 cm to about 4 cm, or about 1.5 cm to about 3 cm. The distal end region 346 of the elongate body 360 can also be shaped with or without a taper. When the catheter advancement element 300 is inserted through the catheter 200, this distal end region 346 is configured to extend beyond and protrude out through the distal-most end 215 of the luminal portion 222 whereas the more proximal region of the body 360 (i.e., the first length described above) remains within the luminal portion 222.

As mentioned, the distal-most end 215 of the luminal portion 222 can be blunt and have no change in the dimension of the outer diameter whereas the distal end region 346 can be tapered providing an overall elongated tapered geometry of the catheter system. The outer diameter of the elongate body 360 also approaches the inner diameter of the luminal portion 222 such that the step-up from the elongate body 360 to the outer diameter of the luminal portion 222 is minimized Minimizing this step-up prevents issues with the lip formed by the distal end of the luminal portion 222 catching on septations, granulations, and tributaries of the venous sinuses, when the distal end region 346 in combination with the distal end region of the catheter 200 bends and curves along within the anatomy. In some implementations, the inner diameter of the luminal portion 222 can be at least about 0.052″ (1.321 mm), about 0.054″ (1.372 mm) and the maximum outer diameter of the elongate body 360 can be about 0.048″ (1.219 mm) such that the difference between them is about 0.006″ (0.1524 mm). In some implementations, the inner diameter of the luminal portion 222 can be about 0.070″ (1.778 mm) and the maximum outer diameter of the elongate body 360 can be about 0.062″ (1.575 mm) such that the difference between them is about 0.008″ (0.2032 mm). In some implementations, the inner diameter of the luminal portion 222 can be about 0.088″ (2.235 mm) and the maximum outer diameter of the elongate body 360 can be about 0.080″ (2.032 mm) such that the difference between them is about 0.008″ (0.2032 mm). In some implementations, the inner diameter of the luminal portion 222 can be about 0.072″ (1.829 mm) and the maximum outer diameter of the elongate body 360 is about 0.070″ (1.778 mm) such that the difference between them is only 2 thousandths of an inch (0.002″/0.0508 mm). In other implementations, the maximum outer diameter of the elongate body 360 is about 0.062″ (1.575 mm) such that the difference between them is about 0.010″ (0.254 mm). Despite the outer diameter of the elongate body 360 extending through the single lumen of the luminal portion 222, the luminal portion 222 and the elongate body 360 extending through it in co-axial fashion are flexible enough to navigate the tortuous anatomy leading to the level of superior sagittal sinus without kinking and without damage. Due to the large size of the distal treatment sites, such as the superior sagittal sinus, it is preferred to deliver a catheter that is as large in inner diameter as possible. The large inner diameter of the catheter is helpful in delivering large aspiration forces to the site of occlusion for aspiration-only treatment without the risk of clogging. The large inner diameter of the catheter is also helpful for the delivery of larger-sized stents. In preferred embodiments, the catheter delivered to the treatment site has a lumen size that is at least about 0.088″.

The dimensions provided herein are approximate and each dimensions may have an engineering tolerance or a permissible limit of variation. Use of the term “about,” “approximately,” or “substantially” are intended to provide such permissible tolerance to the dimension being referred to. Where “about” or “approximately” or “substantially” is not used with a particular dimension herein that that dimension need not be exact.

The length of the tapered distal end region 346 can vary. In some implementations, the length of the distal end region 346 can be in a range of between about 0.50 cm to about 5.0 cm from the distal-most end of the elongate body 360 or between about 1.0 cm to about 4.0 cm, or about 1.5 cm to about 3 cm, or between 2.0 cm to about 2.5 cm. In some implementations, the length of the distal end region 346 varies depending on the inner diameter of the catheter 200 with which the catheter advancement element 300 is to be used. For example, the length of the distal end region 346 can be shorter (e.g. 1.2 cm) for a catheter advancement element 300 sized to be used with a catheter 200 having an inner diameter of about 0.054″ (1.372 mm) and can be longer (e.g. 2.5 cm) for a catheter advancement element 300 sized to be used with a catheter 200 having an inner diameter of about 0.088″ (2.235 mm). The distal end region 346 can be a constant taper from the larger outer diameter of the elongate body 360 (e.g. the distal end of the marker 344b) down to a second smaller outer diameter at the distal-most terminus (e.g. the proximal end of the marker 344a) as shown in FIG. 1C. In some implementations, the constant taper of the distal end region 346 can be from about 0.048″ outer diameter down to about 0.031″ (0.787 mm) outer diameter over a length of about 1 cm. In some implementations, the constant taper of the distal end region 346 can be from 0.062″ (1.575 mm) outer diameter to about 0.031″ (0.787 mm) outer diameter over a length of about 2 cm. In still further implementations, the constant taper of the distal end region 346 can be from 0.080″ (2.032 mm) outer diameter to about 0.031″ (0.787 mm) outer diameter over a length of about 2.5 cm. The length of the constant taper of the distal end region 346 can vary, for example, between 0.8 cm to about 2.5 cm, or between 1 cm and 3 cm, or between 2.0 cm and 2.5 cm. The angle of the taper can vary depending on the outer diameter of the elongate body 360. For example, the angle of the taper can be between 0.9 to 1.6 degrees relative to horizontal. The angle of the taper can be between 2-3 degrees from a center line of the elongate body 360. The length of the taper of the distal end region 346 can be between about 5 mm to 20 mm or about 20 mm to about 50 mm. The tapered portion can taper distally from a first outer diameter that is between 0.048″ and 0.080″ to a second outer diameter that is between 0.031″ up to about 0.048″ over a length of between 0.5 cm and 4.0 cm. The constant taper of the distal tip 346 can taper from a largest, first outer diameter to a second outer diameter. The second outer diameter can be about ½ of the first outer diameter or about 40% of the first outer diameter or about 65% of the first outer diameter. The second outer diameter can be at the distal-most terminus of the elongate body 360.

The elongate body 360 of the catheter advancement element 300 can have a lumen 368 with an inner diameter that does not change over the length of the elongate body even in the presence of the tapering of the distal end region 346. Thus, the inner diameter of the lumen 368 extending through the tubular portion of the catheter advancement element 300 can remain uniform and the wall thickness of the distal end region 346 can decrease to provide the taper. The wall thickness can thin distally along the length of the taper. Thus, the material properties in combination with wall thickness, angle, length of the taper can all contribute to the overall maximum flexibility of the distal-most end of the distal end region 346. The catheter advancement element 300 undergoes a transition in flexibility from the distal-most end towards the snug point where it achieves an outer diameter that is no more than about 0.010″ (0.254 mm) different from the inner diameter of the catheter 200.

The length of the taper can also vary depending on the anatomy of the target region. The distal end region 346 can achieve its soft, atraumatic and flexible characteristic due to a material property other than due to a change in outer dimension to facilitate endovascular navigation to an occlusion in tortuous anatomy. Additionally or alternatively, the distal end region 346 of the elongate body 360 can have a transition in flexibility along its length. The most flexible region of the distal end region 346 can be its distal terminus. Moving along the length of the distal end region 346 from the distal terminus towards a region proximal to the distal terminus. For example, the distal end region 346 can be formed of a material having a Shore material hardness of no more than 35D or about 62A and transitions proximally to be less flexible near where it is formed of a material having a material hardness of no more than 55D and 72D up to the proximal portion 366, which can be a stainless steel hypotube, or a combination of a material property and tapered shape. The materials used to form the regions of the elongate body 360 can include PEBAX (such as PEBAX 25D, 35D, 55D, 69D, 72D) or a blend of PEBAX (such as a mix of and 35D, 25D and 55D, 25D and 72D, 35D and 55D, 35D and 72D, 55D and 72D, where the blend ratios may range from 0.1% up to 50% for each PEBAX durometer), with a lubricious additive compound, such as Mobilize (Compounding Solutions, Lewiston, Maine). In some implementations, the material used to form a region of the elongate body 360 can be Tecothane 62A. Incorporation of a lubricious additive directly into the polymer elongate body means incorporation of a separate lubricious liner, such as a Teflon liner, is unnecessary. A catheter advancement element that is a fully polymeric element without a separate liner or any reinforcement allows for a more flexible element that can navigate the distal anatomy and is less likely to kink Similar materials can be used for forming the distal luminal portion 222 of the catheter 200 providing similar advantages. The flexibility of the distal end region 346 can be achieved by a combination of flexible lubricious materials and tapered shapes. For example, the length of the distal end region 346 can be kept shorter than 2 cm-3 cm, but maintain optimum deliverability due to a change in flexible material from distal-most end 325 towards a more proximal region a distance away from the distal-most end 325. In an implementation, the elongate body 360 is formed of PEBAX (polyether block amide) embedded silicone designed to maintain the highest degree of flexibility. The wall thickness of the distal end of the luminal portion 222 can also be made thin enough such that the lip formed by the distal end of the luminal portion 222 relative to the elongate body 360 is minimized.

The elongate body 360 has a benefit over a microcatheter in that it can have a relatively large outer diameter that is just 0.003″-0.010″ (0.0762 mm-0.254 mm) smaller than the inner diameter of the distal luminal portion 222 of the catheter 200 and still maintaining a high degree of flexibility for navigating tortuous anatomy. When the gap between the two components is too tight (e.g. less than about (0.0762 mm), the force needed to slide the catheter advancement element 300 relative to the catheter 200 can result in damage to one or both of the components and increases risk to the patient during the procedure. The gap results in too tight of a fit to provide optimum relative sliding. When the gap between the two components is too loose (e.g. greater than about 0.010″/0.254 mm), the distal end of the catheter 200 forms a lip that is prone to catch on tributaries feeding into the venous sinuses as well as any granulations and septations that may be present, during advancement through the venous anatomy, such as around the jugular bulb into the sigmoid sinus.

The gap in ID/OD between the elongate body 360 and the distal luminal portion 222 can be in this size range (e.g. 0.003″-0.015″ (0.0762 mm-0.381 mm) or between 0.006″-0.010″ (0.152 mm-0.254 mm)) along a majority of their lengths. For example, the elongate body 360 can have a relatively uniform outer diameter that is between about 0.048″ (1.219 mm) to about 0.080″ (2.032 mm) from a proximal end region to a distal end region up to a point where the taper of the distal end region 346 begins. Similarly, the distal luminal portion 222 of the catheter 200 can have a relatively uniform inner diameter that is between about 0.054″ (1.372 mm) to about 0.088″ (2.235 mm) from a proximal end region to a distal end region. As such, the difference between their respective inner and outer diameters along a majority of their lengths can be within this gap size range of 0.003″ to 0.015″ (0.0762 mm-0.381 mm). The distal end region 346 of the elongate body 360 that is tapered will have a larger gap size relative to the inner diameter of the distal luminal portion 222. During use, however, this tapered distal end region 346 is configured to extend distal to the distal end of the catheter 200 such that the region of the elongate body 360 having an outer diameter sized to match the inner diameter of the distal luminal portion 222 is positioned within the lumen of the catheter 200 such that it can minimize the lip at the distal end of the catheter 200.

The elongate body 360 can be formed of various materials that provide a suitable flexibility and lubricity. Example materials include high density polyethylene, 77A PEBAX, 33D PEBAX, 42D PEBAX, 46D PEBAX, 54D PEBAX, 69D PEBAX, 72D PEBAX, 90D PEBAX, and mixtures thereof or equivalent stiffness and lubricity material. In some implementations, the elongate body 360 is an unreinforced, non-torqueing catheter having a relatively large outer diameter designed to fill the lumen it is inserted through and a relatively small inner diameter to minimize any gaps at a distal-facing end of the device. In other implementations, at least a portion of the elongate body 360 can be reinforced to improve navigation and torqueing (e.g. braided reinforcement layer). The flexibility of the elongate body 360 can increase towards the distal end region 346 such that the distal region of the elongate body 360 is softer, more flexible, and articulates and bends more easily than a more proximal region. For example, a more proximal region of the elongate body can have a bending stiffness that is flexible enough to navigate tortuous anatomy without kinking. If the elongate body 360 has a braid reinforcement layer along at least a portion of its length, the braid reinforcement layer can terminate a distance proximal to the distal end region 346. For example, the distance from the end of the braid to the distal-most end 325 can be about 10 cm to about 15 cm or from about 4 cm to about 10 cm or from about 4 cm up to about 15 cm.

In some implementations, the elongate body 360 can be generally tubular along at least a portion of its length such that it has a single lumen 368 extending parallel to a longitudinal axis of the catheter advancement element 300 (see FIG. 1A-1D). In an implementation, the single lumen 368 of the elongate body 360 is sized to accommodate a guidewire, however use of the catheter advancement element 300 generally eliminates the need for a guidewire lead. Preferably, the assembled system includes no guidewire or a guidewire parked inside the lumen 368 retracted away from the distal opening. Guidewires are designed to be exceptionally flexible so that they deflect to navigate the severe turns of the anatomy. However, many workhorse guidewires have a stiffness along their longitudinal axis and/or are small enough in outer diameter that they find their own paths, for example through a sinus septations, rather than slipping around the septation. Guidewires tend to hang up on venous septae, arachnoid and get redirected into venous tributaries rather than remaining within the larger venous sinuses. In some cases, these guidewires can cause perforations and/or dissections of the sinus wall itself. These guidewire-caused perforations and/or dissections in the venous system can require that the administration of heparin be stopped resulting in exacerbated hypertension, clotting and thrombus, leading to swelling and hemorrhage in the brain and possibly death. Thus, even though the guidewire may have an outer diameter at its distal tip region that is small and very flexible at the distal tip, guidewires typically are incapable of atraumatically probing these structures and pose a risk of perforations with repeated advancements through the venous sinuses. Guidewires do not deflect upon encountering a dense proximal face of the embolus or venous septation. Instead, guidewires embed and penetrate such structures. The catheter advancement element 300 has a softness, taper, and sizing that finds and/or creates space. For example, the catheter advancement element 300 can slide between a portion of an embolus and the sinus wall rather than penetrating through it like a guidewire does. The catheter advancement element 300 deflects away from a septations or arachnoid villi so as to remain within the larger lumen of the sinus. The softness, taper, and sizing of the catheter advancement element 300 allows for it to be repeatedly passed through the venous sinuses without penetrating or taking a detour relative to these structures within the sinus. The distal tip region deflects and passes by these structures so that the catheter system is advanced to an occlusion site. Methods of using the catheter advancement element 300 without a guidewire or with a rescue guidewire 500 parked within the lumen 368 (see FIG. 1D) to deliver a catheter to distal regions of the brain, such as to an occlusion in a superior sagittal sinus, are described in more detail below.

A guidewire can extend through the single lumen 368 generally concentrically from a proximal opening to a distal opening 326 at the distal end 325 of the catheter advancement element 300 through which the guidewire can extend. In some implementations, the proximal opening is at the proximal end of the catheter advancement element 300 such that the catheter advancement element 300 is configured for over-the-wire (OTW) methodologies. In other implementations, the proximal opening is a rapid exchange opening through a wall of the catheter advancement element 300 such that the catheter advancement element 300 is configured for rapid exchange rather than or in addition to OTW. In this implementation, the proximal opening extends through the sidewall of the elongate body 360 and is located a distance away from a proximal tab or luer 364 and distal to the proximal portion 366. The proximal opening can be located a distance of about 10 cm from the distal end region 346 up to about 20 cm from the distal end region 346. In some implementations, the proximal opening can be located near a region where the elongate body 360 is joined to the proximal portion 366, for example, just distal to an end of the hypotube. In other implementations, the proximal opening is located more distally, such as about 10 cm to about 18 cm from the distal-most end of the elongate body 360. A proximal opening that is located closer to the distal end region 346 allows for easier removal of the catheter advancement element 300 from the catheter 200 leaving the guidewire in place for a “rapid exchange” type of procedure. Rapid exchanges can rely on only a single person to perform the exchange. The catheter advancement element 300 can be readily substituted for another device using the same guidewire that remains in position. The single lumen 368 of the elongate body 360 can be configured to receive a guidewire in the range of 0.014″ (0.356 mm) and 0.018″ (0.457 mm) diameter, or in the range of between 0.014″ and 0.022″ (0.356 mm-0.559 mm). In this implementation, the inner luminal diameter of the elongate body 360 can be between 0.020″ and 0.024″ (0.508 mm-0.610 mm). The guidewire, the catheter advancement element 300, and the catheter 200 can all be assembled co-axially for insertion through the working lumen of the guide sheath 400. The inner diameter of the lumen 368 of the elongate body 360 can be 0.019″ to about 0.021″ (0.483 mm-0.533 mm). The distal opening from the lumen 368 can have an inner diameter that is between about 0.018″ to about 0.024″ (0.457 mm-0.610 mm). The distal opening from the lumen 368 can have an inner diameter that is between about 0.016″ to about 0.028″. The distal opening is sized to receive a guidewire that can be a 0.014″ to a 0.024″ guidewire. The single lumen of the flexible elongate body can have an inner diameter of less than about 0.024″.

The region near the distal end region 346 can be tapered such that the outer diameter tapers over a length of about 0.5 cm to about 5 cm, or 1 cm to about 4 cm, or other length as described elsewhere herein. The larger outer diameter can be at least about 1.5 times, 2 times, 2.5 times, or about 3 times larger than the smaller outer diameter. The distal end region 346 can taper along a distance from a first outer diameter to a second outer diameter, the first outer diameter being at least 1.5 times the second outer diameter. In some implementations, the distal end region 346 tapers from about 0.080″ (2.032 mm) to about 0.031″ (0.787 mm). In some implementations, the smaller outer diameter at a distal end of the taper can be about 0.026″ (0.66 mm) up to about 0.040″ (1.016 mm) and the larger outer diameter proximal to the taper is about 0.062″ (1.575 mm) up to about 0.080″ (2.032 mm). Also, the distal end region 346 can be formed of a material having a material hardness (e.g. 62A and 35D) that transitions proximally towards increasingly harder materials having (e.g. 55D and 72D) up to the proximal portion 366. A first segment of the elongate body 360 including the distal end region 346 can be formed of a material having a material hardness of 35D and a length of about 10 cm to about 12.5 cm. The first segment of the elongate body 360 including the distal end region 346 can be formed of a material having a material hardness of 62A and a length of about 10 cm to about 12.5 cm. A second segment of the elongate body 360 can be formed of a material having a material hardness of 55D and have a length of about 5 cm to about 8 cm. A third segment of the elongate body 360 can be formed of a material having a material hardness of 72D can be about 25 cm to about 35 cm in length. The three segments combined can form an insert length of the elongate body 360 from where the proximal portion 366 couples to the elongate body 360 to the terminus of the distal end region 346 that can be about 49 cm in length.

In preferred embodiments it has been found that having a flexible distal tapered probing tip section having a length in the range of 1 cm to 5 cm and that tapers from a proximal outer diameter in the range of 1.58 mm-2.03 mm to a distal outer diameter in the range of 0.66 mm-0.79 mm, the atraumatic tip preferably being radiopaque, that the tapered tip region has a flexibility allowing it to deflect generally away from certain structures present in the venous sinuses so as to carry on within a larger lumen of the venous sinus. The deflection occurs upon advancement of the catheter advancement element through the sinus on encountering a resistance to further axial motion within a flexible sinus having an inner diameter about 7 mm up to about 10 mm. The tip region is arranged to deflect away from structures within a venous sinus, such as arachnoid villi or granulation, venous tributary, or septation within the sinus to find the larger pathway through the sinus. In some instances, the tip region deflects away from the venous structure (e.g., granulation, tributary, or septa) to find the larger pathway through the sinus to distal sites.

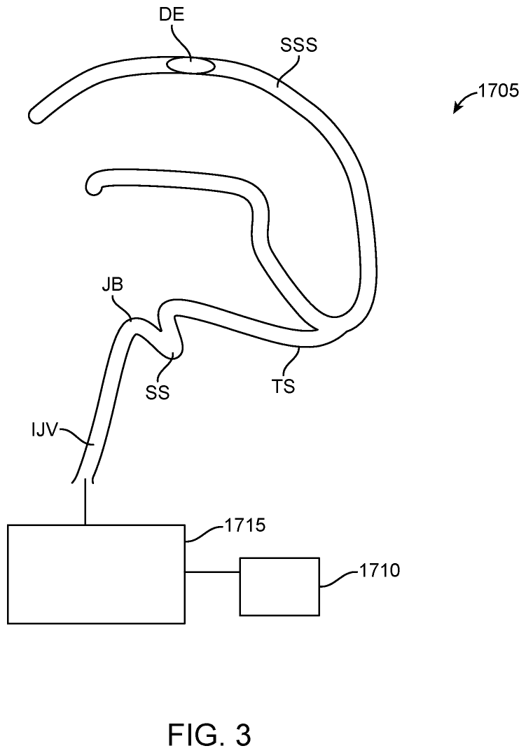

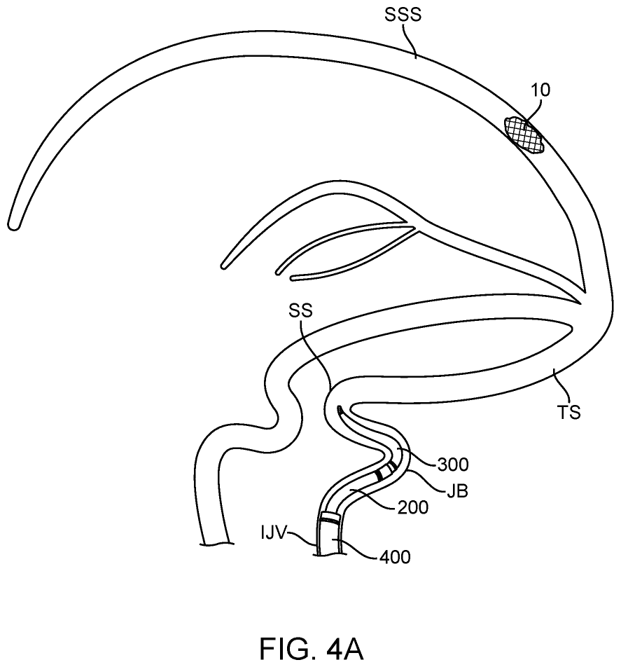

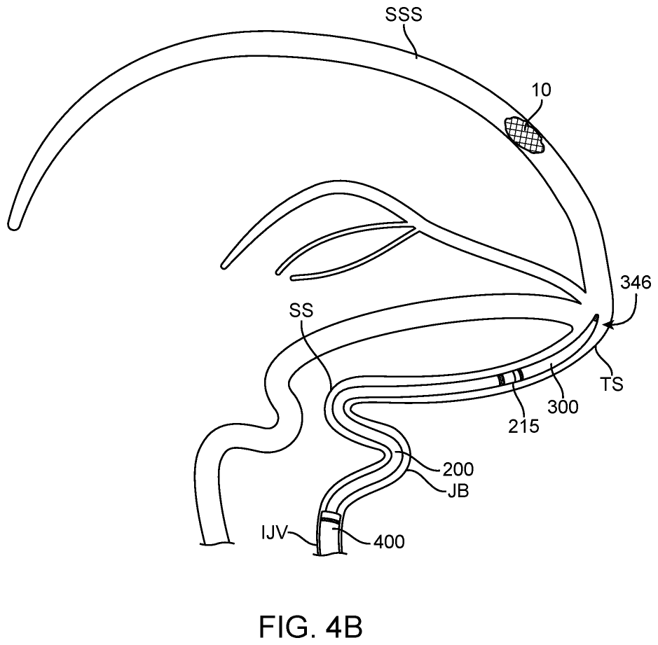

Conventional catheters and guidewires have a tip structure that tend to embed into these structures as opposed to safely probe them to find a space or deflect away from the structure. Guidewires have small outer diameters and flexible distal tips. Despite the small outer diameter and the flexibility, a guidewire tip is incapable of safely probing the venous sinus anatomy according to the methods provided herein. Rather, a guidewire tip construction, particularly when used with a microcatheter that provides a centering effect on the guidewire, results in the guidewire penetrating and embedding into or passing through the structure. FIG. 2A illustrates a conventional guidewire GW extending through and centered by a microcatheter M within a dural venous sinus, for example the superior sagittal sinus. The guidewire GW has a tip region embedded within and penetrating an obstruction 115. FIG. 2B illustrates the tapered distal tip region 346 of a catheter advancement element probing the obstruction 115 so that the tip deflects and slips between the face of the obstruction 115 and the sinus wall. FIG. 2C illustrates the tapered tip distal region 346 preventing advancement of the catheter system in the case of the navigation of the guidewire GW through the space between a chordae and the inner wall of the sinus, rather than the main lumen of the sinus.

The distal end region of the guidewire has a profile that is much smaller compared to the profile of the distal tip region 346 of the catheter advancement element. The outer diameter of the guidewire also stays small moving proximally along its length compared to the catheter advancement element that enlarges to an even larger outer diameter moving proximally just a few centimeters. In turn, the force per unit area for the guidewire is much higher compared to the catheter advancement element. A guidewire used in a venous sinus may have an outer diameter at the distal end that is 0.014″ (0.36 mm) and have a distal-facing contact area that is about 1.50×10⁻⁴ square inch (0.100 mm²). The outer diameter of the distal end of the catheter advancement element can be about 0.031″ (0.79 mm) and the inner diameter of the distal end of the catheter advancement element can be about 0.021″ (0.53 mm). The distal-facing contact area for the catheter advancement element can be about 8.00×10⁻⁴ square inch (0.5 mm²) if the lumen is filled with a column of fluid and/or a guidewire. The distal-facing contact area for the catheter advancement element can be about 4.20×10⁻⁴ square inch (0.27 mm²) for just the annular distal-facing surface without a column of fluid or guidewire within the lumen. Regardless, the force per unit area of the guidewire is significantly greater (i.e., about 2 to 5 times greater) than the force per unit area of the catheter advancement element. The force per unit area of a 0.014″ guidewire for 1 N force is about 6,700 N/square inch (10 N/mm²) whereas the force per unit area of the catheter advancement element is about 1,300 N/square inch (2 N/mm²) to about 2,400 N/square inch (4 N/mm²). The profile of the guidewire, in combination with the force per unit area for the guidewire (and centering effect provided by the microcatheter), creates a higher risk of penetration of the obstruction whether a thrombotic occlusion or an arachnoid villi or septation or other venous sinus structure, such as the sinus wall rather than deflecting upon encountering the structure. The profile of the catheter advancement element including the greater outer diameter as the distal end, the relatively short taper to an even larger outer diameter, and its high flexibility results in the catheter advancement element being incapable of penetrating the structure and instead deflecting away from it upon encountering one within a venous sinus. Guidewires penetrate a granulation or venous wall. The catheter advancement element, in contrast, probes and deflects away from such structures, finds any space and wedges past or into a final resting spot without penetrating the structure or the venous wall.

It is desirable to have a specially constructed tip region to ensure the tip region will deflect, advance with difficulty, or be unable to advance, relative to a structure encountered within the venous sinus, not penetrate it, when encountering it within the sinus. The tip region will deflect until it finds a path or spa or be wedged into a chordae or “false lumen” and cause difficulty with or prevent advancement. This is achieved by having a sufficient degree of flexibility of the fully polymeric (i.e., having no reinforcement layer) distal tip region that includes a taper over a length so that the tip region wedges atraumatically or deflects readily upon coming into contact with the structure, whether that structure is a chordae in the wall of the sinus, a tributary feeding the venous sinus, a granulation or septation. The flexibility and shape of the tapered tip region results in the tip region, which is protruding from the catheter during advancement through the venous sinus, passing through the sinus until the tip region encounters a true proximal face of the obstruction being treated, which can be an embolus, thrombotic material a false lumen formed from the wall irregularities, such as the chordae willisii, or a stenosis of the sinus. The tip region is then wedged into or deflects away from the organized or dense structure so that, for example, it wedges between the obstruction or chordae and the venous sinus wall. The tip region is constructed to find the path of least resistance in an atraumatic manner without being so flexible or prone to bending that it folds over onto itself and cannot be advanced. Thus, the user can determine in an atraumatic manner if he/she is in the area of an obstruction, partial obstruction, or false lumen, and can act or react accordingly to position and advance the catheter in the desired manner.

The distal-most tip of the tip region can have a smooth, relatively rounded shape having a low friction outer surface that tends to encourage deflection of the tip region relative to venous sinus structures. The distal tip can also be radiopaque due to embedding a material within the polymer as described in more detail below.

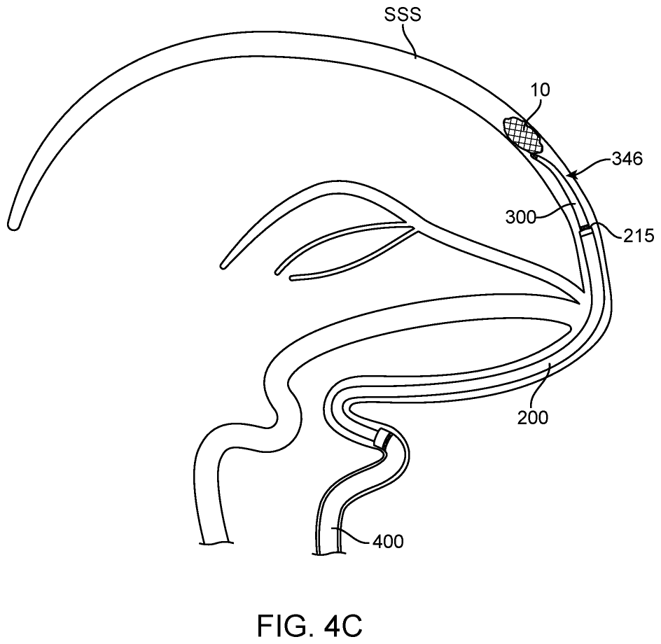

One of skill in the art can “tune” the distal tip region to have one or more properties to achieve the novel requirements set out herein. However, because the requirements are so unusual, it may be useful to measure the properties of the distal tip region using a test rig 1705. For example, FIG. 3 illustrates an implementation of a test rig 1705 in schematic. The test rig 1705 can include a 3D printed model of clear silicone material based on a CT/MRI scan data of an actual human patient that is configured to be connected to a pump 1710 for delivering a liquid from a source 1715 to simulate the endovascular environment. The vessels modeled by the test rig 1705 can vary, including, but not limited to femoral vein, external iliac vein, renal vein, inferior and superior vena cava, internal jugular vein, jugular bulb, sigmoid sinus, transverse sinus, superior sagittal sinus, and others. FIG. 3 illustrates the internal jugular vein (IJV), jugular bulb (JB), sigmoid sinus (SS), transverse sinus (TS), and superior sagittal sinus (SSS). The intracranial veins of the test rig 1705 can include various sized vessels including the one or more obstructions within the sinuses, such as a stenosis of the venous sinus, arachnoid villus, septation, and tributaries leading to the sinuses. FIG. 3 shows a dummy embolus DE formed of a suitable material can be positioned within the vessel model, for example, within the superior sagittal sinus SSS as shown in FIG. 3, to simulate an actual obstruction. The material can include a moldable, compressible polymeric material that can be compressed into a small plug shape suitable for insertion into a vessel of interest on the test rig 1705. The larger sinuses of the test rig 1705 can have an internal diameter of about 10-11 mm that decreases down to about 5 mm ID. The model sinus containing the dummy embolus DE can have an inner diameter of about 8 mm and can taper along its length although smaller or larger vessels can also be used. The dummy embolus DE can be compressed into a plug that has a maximum outer diameter that substantially matches the inner diameter of the vessel being obstructed by the dummy embolus DE. The material of the dummy embolus DE can have an outer diameter prior to being compressed that is about 12 mm to about 17 mm and a length of about 5 mm. The length of the dummy embolus DE can increase upon being compressed into the smaller diameter plug or can be trimmed after compressing to have a particular length. The dummy embolus DE once compressed can be positioned within the target sinus. The dummy embolus DE once positioned in the target sinus can fully or partially block fluid flow through the model and past the dummy embolus DE. The dummy embolus DE can have a density at its proximal face (“proximal” being relative to the direction of catheter advancement) that is comparable to a typical embolus treated in this part of the venous sinus and used to observe the degree of deflection a distal tip region 346 of a catheter advancement element positioned distal to the aspiration catheter 200 being advanced. The material of the dummy embolus DE can be selected so as to have different consistencies to emulate the different types of obstructions that might be encountered. The test rig 1705 provides a way to assess whether the distal tip region 346 of the catheter advancement element will deflect or embed within the dummy embolus DE. The test rig 1705 can also assess the impact of a guidewire positioned within the lumen of the catheter advancement element, for example so the distal end of the guidewire is positioned proximal to the distal opening from the lumen, on the deflection of the distal tip region 346 upon encountering the different dummy emboli DE. The dummy embolus DE can be formed of any of a variety of materials to resemble the generally loose, soft, high volume occlusions that may form in a venous sinus (e.g., noise putty slime) versus a more focal, dense type of material. Any of a variety of configurations are considered. Those of skill in the art may have alternative test rigs incorporating alternative real or synthetic obstruction test subjects including other materials shaped to form an obstruction or other venous structure.

The catheter advancement element 300 can incorporate a reinforcement layer. The reinforcement layer can be a braid or other type of reinforcement to provide or improve the torqueability of the catheter advancement element 300 and help to bridge the components of the catheter advancement element 300 having such differences in flexibility. The reinforcement layer can bridge the transition from a rigid, proximal portion 366 to the flexible elongate body 360. In some implementations, the reinforcement layer can be a braid positioned between inner and outer layers of PEBAX. The reinforcement layer can terminate a distance proximal to the distal end region 346. The distal end region 346 can be formed of a material having a material hardness of at most about 35D. The first segment can be unreinforced polymer having a length of about 4 cm up to about 12.5 cm without metal reinforcement. The third segment of the elongate body 360 located proximal to the first segment can include the reinforcement layer and can extend a total of about 37 cm up to the unreinforced distal segment. A proximal end region of the reinforcement layer can overlap with a distal end region of the proximal portion 366 such that a small overlap of hypotube and reinforcement exists near the transition between the proximal portion 366 and the elongate body 360.

The catheter advancement element 300 can include a distal portion and a proximal segment, such as a hypotube. An intermediate segment of the catheter advancement element 300 can include an unreinforced polymer having a first durometer. The distal portion of the catheter advancement element can include a polymer that is different from the unreinforced polymer of the intermediate segment. The polymer of the distal portion can have a durometer that is less than the durometer of the unreinforced polymer of the intermediate segment.

An entry port for a procedural guidewire can be positioned a distance away from the distal-most end of the elongate body 360. In some implementations, the entry/exit port can be about 18 cm from the distal-most end creating a rapid exchange wire entry/exit segment. The outer diameter of the elongate body 360 within the first two segments can be about 0.080″-0.082″ (2.032 mm-2.083 mm) whereas the third segment proximal to this rapid exchange wire entry/exit segment can have a step-down in outer diameter, such as about 0.062″-0.064″ (1.575 mm-1.626 mm).

The tubular portion of the catheter advancement element 300 can have an outer diameter that has at least one snug point. A difference between the outer diameter at the snug point and the inner diameter of the lumen at the distal end of the distal, catheter portion can be no more than about 0.015″ (0.381 mm), or can be no more than about 0.010″ (0.254 mm). The at least one snug point of this tubular portion can be a point along the length of the tubular portion. The at least one snug point of this tubular portion can have a length that is at least about 5 cm up to about 50 cm, including for example, at least about 6 cm, at least about 7 cm, at least about 8 cm, at least about 9 cm, at least about 10 cm, at least about 11 cm, or at least about 12 cm up to about 50 cm. This length need not be uniform such that the length need not be snug along its entire length. For example, the snug point region can include ridges, grooves, slits, or other surface features.

In other implementations, the entire catheter advancement element 300 can be a tubular element configured to receive a guidewire through both the proximal portion 366 as well as the elongate body 360. For example, the proximal portion 366 can be a hypotube or tubular element having a lumen that communicates with the lumen 368 extending through the elongate body 360 (shown in FIG. 1C). In some implementations, the proximal portion 366 can be a skived hypotube of stainless steel coated with PTFE having an outer diameter of 0.026″ (0.660 mm). In other implementations, the outer diameter can be between 0.024″ (0.610 mm) and 0.030″ (0.762 mm). In some implementations, such as an over-the-wire version, the proximal portion 366 can be a skived hypotube coupled to a proximal hub or luer 364. The proximal portion 366 can extend eccentric or concentric to the distal luminal portion 222. The proximal portion 366 can be a stainless steel hypotube. The proximal portion 366 can be a solid metal wire that is round or oval cross-sectional shape. The proximal portion 366 can be a flattened ribbon of wire having a rectangular cross-sectional shape. The ribbon of wire can be curved into a circular, oval, c-shape, or quarter circle, or other cross-sectional shape along an arc. The proximal portion 366 can have any of variety of cross-sectional shapes whether or not a lumen extends therethrough, including a circular, oval, C-shaped, D-shape, or other shape. In some implementations, the proximal portion 366 is a hypotube having a D-shape such that an inner-facing side is flat and an outer-facing side is rounded. The rounded side of the proximal portion 366 can be shaped to engage with a correspondingly rounded inner surface of the sheath 400. The hypotube can have a lubricious coating, such as PTFE or other lubricious polymer, covering the hypotube. The hypotube can have an inner diameter of about 0.021″ (0.533 mm), an outer diameter of about 0.0275″ (0.699 mm), and an overall length of about 94 cm providing a working length for the catheter advancement element 300 that is about 143 cm. Including the proximal luer 364, the catheter advancement element 300 can have an overall length of about 149 cm. In some implementations, the hypotube can be a tapered part with a length of about 100 mm, starting proximal with a thickness of 0.3 mm and ending with a thickness of 0.10 mm to 0.15 mm. In still further implementations, the elongate body 360 can be a solid element coupled to the proximal portion 366 having no guidewire lumen.

The proximal portion 366 is shown in FIG. 1A as having a smaller outer diameter compared to the outer diameter of the elongate body 360. The proximal portion 366 need not step down in outer diameter and can also have the same outer diameter as the outer diameter as the elongate body 360. For example, the proximal portion 366 can incorporate a hypotube or other stiffening element that is coated by one or more layers of polymer resulting in a proximal portion 366 having substantially the same outer diameter as the elongate body 360.

At least a portion of the solid elongate body 360, such as the elongate distal end region 346, can be formed of or embedded with or attached to a malleable material that skives down to a smaller dimension at a distal end. The distal end region 346 can be shaped to a desired angle or shape similar to how a guidewire may be used. The malleable length of the elongate body 360 can be at least about 1 cm, 3 cm, 5 cm, and up to about 10 cm, 15 cm, or longer. In some implementations, the malleable length can be about 1%, 2%, 5%, 10%, 20%, 25%, 50% or more of the total length of the elongate body 360. In some implementations, the catheter advancement element 300 can have a working length of about 140 cm to about 143 cm and the elongate body 360 can have an insert length of about 49 cm. The insert length can be the PEBAX portion of the elongate body 360 that is about 49.5 cm. As such, the malleable length of the elongate body 360 can be between about 0.5 cm to about 25 cm or more. The shape change can be a function of a user manually shaping the malleable length prior to insertion or the distal end region 346 can be pre-shaped at the time of manufacturing into a particular angle or curve. Alternatively, the shape change can be a reversible and actuatable shape change such that the distal end region 346 forms the shape upon activation by a user such that the distal end region 346 can be used in a straight format until a shape change is desired by the user. The catheter advancement element 300 can also include a forming mandrel extending through the lumen of the elongate body 360 such that a physician at the time of use can mold the distal end region 346 into a desired shape. As such, the moldable distal end region 346 can be incorporated onto an elongate body 360 that has a guidewire lumen.

The elongate body 360 can extend along the entire length of the catheter 200, including the distal luminal portion 222 and the proximal extension 230 or the elongate body 360 can incorporate the proximal portion 366 that aligns generally side-by-side with the proximal extension 230 of the catheter 200. The proximal portion 366 of the elongate body 360 can be positioned co-axial with or eccentric to the elongate body 360. The proximal portion 366 of the elongate body 360 can have a lumen extending through it. Alternatively, the portion 366 can be a solid rod or ribbon having no lumen.

Again with respect to FIGS. 1A-1D, like the distal luminal portion 222 of the catheter 200, the elongate body 360 can have one or more radiopaque markers 344 along its length. The one or more markers 344 can vary in size, shape, and location. One or more markers 344 can be incorporated along one or more parts of the catheter advancement element 300, such as a tip-to-tip marker, a tip-to-taper marker, an RHV proximity marker, a Fluoro-saver marker, or other markers providing various information regarding the relative position of the catheter advancement element 300 and its components. In some implementations and as best shown in FIGS. 1C-1D, a distal end region can have a first radiopaque marker 344a and a second radiopaque marker 344b can be located to indicate the border between the tapering of the distal end region 346 and the more proximal region of the elongate body 360 having a uniform or maximum outer diameter. This provides a user with information regarding an optimal extension of the distal end region 346 relative to the distal end of the luminal portion 222 to minimize the lip at this distal end of the luminal portion 222 for advancement through tortuous anatomy. In other implementations, for example where the distal end region 346 is not necessarily tapered, but instead has a change in overall flexibility along its length, the second radiopaque marker 344b can be located to indicate the region where the relative flexibilities of the elongate body 360 (or the distal end region 346 of the elongate body 360) and the distal end of the luminal portion 222 are substantially the same. The marker material may be a platinum/iridium band, a tungsten, platinum, or tantalum-impregnated polymer, or other radiopaque marker that does not impact the flexibility of the distal end region 346 and elongate body 360. In some implementations, the radiopaque markers are extruded PEBAX loaded with tungsten for radiopacity. In some implementations, the proximal marker band can be about 2.0 mm wide and the distal marker band can be about 2.5 mm wide to provide discernable information about the distal end region 346.

The catheter advancement element 300 can be positioned through the lumen of the catheter 200 such that its distal end region 346 extends just beyond a distal-most end 215 of the catheter 200. The distal end region 346 of the catheter advancement element 300 eliminates the stepped transition between the inner member and the outer catheter 200 thereby avoiding issues with catching on branching vessels within the region of the vasculature such that the catheter 200 may easily traverse the multiple angulated turns of the dural venous sinuses. The optimum relative extension, for example, can be the distal end region 346 of the elongate body 360 extending just distal to a distal-most end 215 of the catheter 200. A length of the distal end region 346 extending distal to the distal-most end 215 of the catheter 200 during advancement can be between 0.5 cm and about 4 cm. This juxtaposition can be a locked engagement with a mechanical element or simply by a user holding the two components together. The mechanical locking element can be a fixed or removable mechanical element configured to connect to one or more of the catheter 200, the catheter advancement element 300, and the guidewire 500. The mechanical locking element can be slidable along at least a length of the system components when coupled so that the mechanical attachment is adjustable. The mechanical locking element can be a disposable feature or reusable for connecting to at least a portion of the shaft or a more proximal portion of the component, such as the luer or hub at a proximal end of the component. In some implementations, the mechanical locking element can be clamped onto the catheter and the catheter advancement element in a desired relative position so that the two can be advanced together without the relative position being inadvertently changed. The relative position can be changed, if desired, while the mechanical locking element is clamped onto the catheter and the catheter advancement element. The mechanical locking element can be additionally clamped onto a region of the guidewire extending through the catheter advancement element such that the relative position of all three components can be maintained during advancement until a relative sliding motion is desired. In still further implementations, the clamping position of the mechanical locking element can be changed from engaging with a first combination of components (e.g., the catheter, catheter advancement element, and the guidewire) to a different combination of components (e.g., the catheter advancement element and the guidewire) depending on what phase of the method is being performed. In still further implementations, the guidewire is held fixed relative to the catheter advancement element via a rotating hemostatic valve coupled to the proximal hub and the catheter advancement element is held fixed to the catheter by a separate mechanical locking element. Whether the relative position of the components is fixed by a mechanical element, a combination of mechanical elements, or by a user, the proximal portions of each of the catheter 200 and the catheter advancement element 300 (and the guidewire, if present) are configured to be held at a single point by a user. For example, where the catheter and catheter advancement element are advanced and/or withdrawn manually, the single point can be between just a forefinger and thumb of the user.

The components can be advanced together with a guidewire, over a guidewire pre-positioned, or without any guidewire at all. In some implementations, the guidewire can be pre-assembled with the catheter advancement element 300 and catheter 200 such that the guidewire extends through a lumen of the catheter advancement element 300, which is loaded through a lumen of the catheter 200, all prior to insertion into the patient. The pre-assembled components can be simultaneously inserted into the sheath 400 and advanced together up through and past the turns into the venous sinuses. A guidewire may be located within the lumen 368 of the catheter advancement element 300 and parked proximal of the tapered distal end region 346 or proximal of the distal tip for potential use in the event the catheter advancement element without a guidewire does not reach the target location. For example, a distal tip of the guidewire can be positioned about 5 cm to about 40 cm, or about 20 cm to about 30 cm proximal of the distal end region 346 of the catheter advancement element 300. At this location the guidewire does not interfere with the performance or function of the catheter advancement element. The guidewire can be positioned within the lumen of the catheter advancement element such that the distal end of the guidewire is within the catheter advancement element during the step of advancing the assembled system of devices together and is extendable from the catheter advancement element out the distal opening 326 when needed for navigation. In one example, a rescue guidewire is parked within the lumen of the catheter advancement element with a distal end of the guidewire about 0 cm to about 40 cm proximal or about 5 cm to about 35 cm proximal or about 7 cm to about 30 cm of the distal end of the catheter advancement element, preferably about 10 cm proximal of the distal end of the catheter advancement element. The guidewire at this parked position can provide additional support for the proximal portion of the system without affecting the flexibility and performance of the distal portion of the system.

FIG. 1D illustrates a rescue guidewire 500 parked within the lumen 368 of the catheter advancement element 300. In some implementations, the distal end of the guidewire 500 can be positioned inside the lumen 368 approximately flush with (0 cm) the distal opening 326 of the catheter advancement element during advancement through the venous sinus. In some implementations, the distal end of the guidewire 500 can be positioned inside the lumen 368 a distance proximal from the distal opening 326 of the catheter advancement element during advancement through the venous sinuses. The distance between the distal end of the parked guidewire 500 and the distal opening 326 can be at least about 1.5 cm, at least about 3 cm, at least about 5 cm, at least about 10 cm, at least about 15 cm, at least about 20 cm, at least about 25 cm, at least about 30 cm, up to about 40 cm proximal to the distal opening 326. Positioning the distal end of the parked guidewire 500 closer to the distal opening 326 of the catheter advancement element (e.g., 1.5 cm to about 3 cm proximal to the distal opening 326) such that it extends within the lumen 368 of the distal tip region 346 can support or stiffen the distal tip region 346 and also support the more proximal regions of the catheter advancement element 300. For example, a surgeon may desire to position the distal end of the guidewire closer to the distal opening 326 to increase stiffness of the distal end region of the catheter advancement element 300. Positioning the distal end of the parked guidewire 500 further away from the distal opening 326 of the catheter advancement element (e.g., greater than 5 cm up to about 40 cm proximal to the distal opening 326, preferably about 10 cm) avoids changing the flexibility characteristics of the distal tip region 346 while still supporting the more proximal regions of the catheter advancement element 300.

The tubular portion 360 of the catheter advancement element 300 can have a radiopaque marker band embedded within or positioned over a wall of the tubular portion 360 near the distal end region 346. A first radiopaque marker band 344a can be found at the distal end of the tapered distal end region 346 and a second radiopaque marker band 344b can be found at the proximal end of the tapered distal end region 346. The proximal radiopaque marker band 344b can have a proximal edge, a distal edge, and a width between the proximal and distal edges. When in the advancement configuration, the proximal edge of the radiopaque marker band 344b can align substantially with the distal end of the distal, catheter portion 222 such that the radiopaque marker band 344b remains external to the lumen 223 of the distal, catheter portion 222. At least a portion of the radiopaque marker band 344b can be positioned at the snug point, or the point of the catheter advancement element 300 where the outer diameter is no more than about 0.010″ (0.254 mm), preferably between about 0.006″ and 0.008″ (0.152 mm-0.203 mm) smaller than the inner diameter of the catheter 200 it is positioned within. The at least one snug point of the tubular portion 360 can be located proximal to the distal end region 346 and can be where the taper of the distal end region 346 substantially ends. This allows for full extension of the tapered distal end region 346 outside the distal end of the catheter 200 and the snug point aligned substantially within the distal opening 231 from the lumen 223 of the distal, catheter portion 222 thereby minimizing any distal-facing lip that might be created by the catheter 200. The snug point can be located along at least a portion of a length of the outer diameter of the tubular portion 360 that has a length of at least about 5 cm up to about cm, the outer diameter being substantially uniform or non-uniform. Additionally, the radiopaque marker bands 344a, 344b can be visible to a user without fluoroscopy, for example, prior to inserting the catheter system into the patient. The marker bands 344a, 344b can form a contrasting color visible to a user compared to a color of the polymer of the flexible elongate body, such as a black band relative to a white color of the polymer. The marker bands 344a, 344b can be useful in achieving a particular relative extension of the catheter advancement element 300 to the catheter 200 prior to insertion of the devices into an RHV.

The use of the catheter advancement element 300 with the tapered distal end region 346 allows for delivery of large bore aspiration catheters, even full-length “over-the-wire” catheters or catheters, such as those described herein having a proximal extension. The catheter advancement element 300 can include a pair of radiopaque markers 344a, 344b configured to aid the operator in delivery of the system. The distal marker 344a near the distal-most end 325 of the catheter advancement element 300 can be differentiated from the distal marker 224a on the catheter 200 by its characteristic appearance under fluoroscopy as well as by simply jogging back and forth the atraumatic catheter advancement element 300 to understand the relationship and positioning of the catheter advancement element 300 relative to the catheter 200. The second marker 344b on the catheter advancement element 300 that is proximal to the distal-most tip marker 344a can delineate the taper of the distal end region 346, i.e. where the outer diameter of the catheter advancement element 300 has a sufficient size to reduce the “lip” of the transition between the catheter advancement element 300 and the catheter 200 through which it is inserted and configured to deliver. The markers aid in positioning the catheter advancement element 300 relative to the distal end 215 of the aspiration catheter 200 such that the tip 215 of the catheter 200 is aligned with the taper of the catheter advancement element 300 and the best alignment is facilitated.

The relationship between the distal tip marker 224 of the aspiration catheter 200 is at or ideally just proximal to the taper marker 344b of the catheter advancement element 300 (i.e. the proximal marker identifying the start of the taper) is identifiable with the tandem marker system. The paired elements 224, 344b are in a “tip-to-taper” position. The relative extension between the catheter advancement element 300 and the catheter 200 can be adjusted at the insertion of the system into the RHV. However, the relative extension can become altered with advancement through the sheath or guide catheter. As the system exits the guide catheter, the aspiration catheter 200 and the catheter advancement element 300 can be adjusted to that the tip-to-taper position is assumed as the system traverses the often tortuous proximal anatomy (e.g. the jugular bulb into the sigmoid sinus) towards more distal targets. The system of the aspiration catheter 200 and the catheter advancement element 300 can be locked into their relative extension so that the juxtaposition of the catheter advancement element 300 and the aspiration catheter 200 is maintained. As the aspiration catheter 200 is visualized within the sheath distal end or even slightly beyond the distal end of the sheath, the catheter advancement element 300 can be adjusted to assume the proper position relative to the catheter before advancement resumes. The optimum relative extension between the distal marker 224 of the catheter 200 to the taper marker 344b on the catheter advancement element 300 can be maintained through as much of the anatomy as possible to maximize the delivery capability of the catheter advancement element 300 to navigate both tortuosity and to avoid tributaries of the venous sinuses. Once a desired site is reached, the catheter advancement element 300 can be held fixed and the aspiration catheter 200 advanced over the catheter advancement element 300 towards the obstruction.

The catheter advancement element 300 is designed specifically such that the catheter 200 can be delivered without a need for a guidewire. This ability to deliver the catheter 200 without a guidewire (or with a guidewire located within the lumen 368 of the catheter advancement element 300 and parked proximal of the tapered distal end region 346 and/or proximal of the distal opening 326 for potential use) is based, in part, upon the smooth transitions between the outer diameter of the catheter advancement element 300 and the catheter 200 as well as the smooth transition in flexibility between the two. When the catheter advancement element 300 is bent into an arc of greater than 180 degrees, the softness and flexibility creates a smooth arc without severe bends or kinks in the geometry of the catheter. Thus, the catheter advancement element 300 seeks the larger lumens and goes where the majority of blood flow goes as opposed to into the smaller tributaries. The distal end region 346 of the catheter advancement element 300 can facilitate a strong preference to seek out the larger vessels during advancement into the distal vessels. This propensity to stay within the main channel allows for the advancement of large bore catheters without the aid of a guidewire.

Standard neurovascular intervention, and nearly all endovascular intervention, is predicated on the concept that a guidewire leads a catheter to a target location. The guidewires are typically pre-shaped and often find side-branches of off-target locations where the guidewire will bunch or prolapse causing time-consuming nuisances during interventions that often require repeated redirection of the guidewire by the operator to overcome. In addition, this propensity of a guidewire to enter tributaries and septations can be problematic, particularly in the transverse, superior sagittal sinus (SSS), and sagittal sinuses. Guidewires are typically 0.014″ to 0.018″ (0.356 mm-0.457 mm) will find and often traumatize septations and granulations and tributaries that accommodate this size, which can lead to small bleeds or dissections and further occlusions. In a sensitive area like the brain these events can be catastrophic. The tendency of a guidewire to bunch and prolapse can also cause a leading edge to the guidewire that can be advanced on its own or as part of a triaxial system to create dissection planes and traumatize small vessels. Guidewires are also designed to cross structures, such as an embolus, primarily for the purpose of securing the guidewire to provide support for delivery of a catheter over the guidewire. However, crossing an embolus with the guidewire can increase a risk of dislodging embolic debris, which can create additional occlusion sites. Guidewires also increase the risk of perforations.

In contrast, the catheter advancement element 300 described herein preferentially stays in the larger lumen of a dural venous sinus. The catheter advancement element 300 delivers to the largest lumen within the anatomy even in light of the highly tortuous anatomy and curves being navigated. The catheter advancement element 300 can preferentially take the larger lumen at a tributary while also maintaining the general direction and angulations of the sinus avoiding perforations or getting caught within granulations and septatations.

Navigation using the catheter advancement element can provide maximal deliverability with minimal vascular trauma. Catheters can cause “razoring” effects in a curved vessel because the blunt end of a large bore catheter can tend to take the greater curve in rounding a vessel when pushed by the operator. This blunt end can gouge or “razor” the greater curve with its sharp edge increasing the risk for dissection along an anatomic plane within the multilayered mid- or large-sized artery or vein (see, e.g. Catheter Cardiovasc. Interv. 2014 February; 83(2):211-20). The catheter advancement element can serve to minimize the edge of these catheters. Positioning the catheter advancement element within the lumen of the large-bore catheter such that the taper marker of the catheter advancement element is aligned optimally with the distal tip marker of the catheter minimizes the edge and thereby eliminates “razoring” as the large-bore catheter is advanced through turns of the vessel. This is particularly useful for the venous sinuses. The specifics of the catheter advancement element in proper alignment within the large bore catheter (the “tip-to-taper” position noted by the distal tip marker) relative to the taper marker of the catheter advancement element maximize the likelihood that razoring and hang-up on the venous tributaries and other structures within the sinuses are avoided during manual advancement of the catheter system. In the arterial anatomy, the catheter advancement element can be helpful navigating known anatomical landmarks known to cause difficulty in reading distal sites, such as the ophthalmic artery. The taper marker of the catheter advancement element can be positioned at or past the take-off of the ophthalmic artery to minimize these deleterious effects and allows the large-bore catheter to pass the ophthalmic artery without incident. In a relatively straight segment, which is common after passing the siphon, the large-bore catheter can be advanced over the catheter advancement element, which serves still as a guiding element to the target. The venous sinus anatomy is less standard and predictable patient-to-patient. Unlike the known branches in the arterial side of the cerebral circulation (e.g., ophthalmic branch off the middle cerebral artery just past the siphon), the location of the tributaries feeding the different sinuses can vary significantly from patient to patient. A surgeon is not able to predict the location of the various tributaries so as to modify the advancement of the catheter system through this segment of the anatomy. Similarly, the location of arachnoid granulations or septations within the venous sinuses are unpredictable and non-standard.

Conventional techniques to treat thrombotic occlusions whether with a stent retriever, aspiration techniques, or a combination of the two, require crossing the target occlusion with a guidewire and a microcatheter. Crossing of the occlusion with a guidewire and then microcatheter can create fragmentation emboli, which can be friable and thrombotic in nature creating particulate that can be released downstream. The aspiration techniques described herein allow for an embolus to be removed en toto without any crossing of the embolus with any device. The systems described herein need not incorporate a guidewire or microcatheter. And, if a guidewire and microcatheter are used, they need not be advanced to cross the target embolus. Thus, the systems described herein can incorporate relatively large bore catheters that are delivered without disturbing the target embolus, reducing the risk for stroke and fragmentation of the embolus, and having improved efficiency. The catheter advancement element allows for multiple deliveries of the aspiration catheter with less risk of perforation and trauma within the venous sinuses. Additionally, the systems described herein are single-operator systems allowing the operator to work at a single RHV and, in the case of spined components, can manipulate all the elements being used to navigate the anatomy with single-handed “pinches.” This can be referred to as “monopoint.”

The catheter advancement element allows for safer and more efficient delivery of large-bore aspiration catheters to distal sites of the venous sinuses. Large-bore aspiration catheters are particularly useful for removing thrombotic material, particularly where the anatomy is very large (e.g., superior sagittal sinus). Catheter inner diameter can be maximized for treating these locations to obtain more beneficial fluid dynamics for aspirating and removing thrombotic material. The pathology of the venous sinuses need not be thrombotic to take advantage of the large-bore catheter delivery. The safe and efficient delivery of the large bore catheter made possible by the catheter advancement element can act as support catheters for the delivery of correspondingly large stents. Venous sinuses, which can be as large as 10-11 mm, require correspondingly large stents in order to treat venous stenosis. Carotid stents, such as the WALLSTENT (Boston Scientific) or PRECISE (Cordis), can be delivered to the venous sinuses, but their large size makes delivery particularly challenging. The catheter advancement element safely and efficiently delivers a large bore catheter (e.g., at least 0.088″) directly to the distal venous sinuses that can then be used as a support catheter for large stent delivery systems (with or without aspiration). The catheter advancement element reduces the risk of wall perforation, particularly compared to a microcatheter-centered guidewire, so that the large bore catheter having a size that approaches the size of the sinus being treated can be delivered more safely.

As mentioned above, angiography is the industry standard for imaging cardiovascular anatomy within the body prior to and during a catheterization procedure. Generally, angiography involves injection of contrast media through the arterial system and use of x-ray fluoroscopic image guidance to visualize the occlusion and/or advancement of the catheter systems toward the occlusion site. The angiogram provides a “roadmap” for navigating the vasculature. Because contrast is typically injected from the arterial side, a complete occlusion within the venous sinus prevents the infiltration of contrast into the venous anatomy. Thus, the “road” that must be navigated by a catheter to reach a venous occlusion is typically invisible on angiogram and navigation to the treatment site performed blind. The catheter advancement element allows for the safe and efficient delivery of large-bore catheters to distal sites of the venous sinus anatomy without angiographic navigation.

The distal end region 346 of the catheter advancement element 300 can be tapered, soft and flexible so that it can be used to locate a desired location relative to the obstruction for application of aspiration by the aspiration catheter 200. The softness, tapering, and sizing of the catheter advancement element 300 distal end region 346 allows for the distal most end of the distal end region 346 to navigate through the venous sinuses and probe the obstruction without perforation of the venous wall or getting caught in granulations or septa. In some implementations, the distal end region 346 can find and/or create space in or beside obstruction or slide between at least a portion of the obstruction and the vessel wall. Unlike a guidewire, the catheter advancement element 300 is unlikely to perforate the wall or cross the obstruction due to the extremely flexible distal tip region and the tapered walls of the distal tapered region 346. Instead, the tapered distal region 346 finds a natural resting point or stopping point where further advancement is prevented or difficult. If the tapered distal end region 346 of the catheter advancement element is advanced beyond this natural stopping point and further advancing pressure is applied, the catheter advancement element can begin to buckle and/or prolapse giving the feedback that the desired advancement has been achieved. If contrast is able to penetrate the obstruction, this buckling between the markers 344a and 344b can be seen angiographically as the marker 344b moving distally without corresponding motion of the marker 344a. Alternatively, the contact between the tapered distal end region 346 and the obstruction can provide feedback, for example, tactile feedback to a user handling the tools manually, that the natural resting place has been reached.

“Crossing the embolus” or “crossing the occlusion” as used herein means that at least some portion of the device crosses to a distal side of the embolus or occlusion relative to the direction or advancement or site of insertion. The limits of an embolus or occlusion can be difficult to assess. Thus, crossing the embolus or occlusion includes at least some portion of the device passing an enlarged region of the occlusion or embolus even though that portion may not be fully distal to the full limit of the lesion. Crossing increases the risk of embolic material being knocked loose from the occlusion or embolus and traveling to create new occlusion sites. The catheter advancement element 300 can be advanced as far as possible without buckling of the catheter advancement element 300. Instead of crossing the occlusion, the catheter advancement element can interrogate the treatment site to locate a proximal face of the occlusion while maintaining structural integrity of both the catheter advancement element 300 and the occlusion.

The occlusive material can be captured at, within, or through the distal end 215 of the aspiration catheter 200 while applying aspiration from an external aspiration source, for example, an aspiration source coupled to the RHV 434 of the base sheath 400. The occlusive material can also be captured during proximal withdrawal of the catheter advancement element 300. As mentioned above, the tubular portion of the catheter advancement element can have an outer diameter that forms a relatively snug tolerance with the aspiration catheter. A difference between the outer diameter at a snug point of the tubular portion and the inner diameter of the lumen at the distal end of the distal, catheter portion can be no more than about 0.015″, for example, between about 0.003″ and 0.015″. This relatively large outer diameter of the catheter advancement element 300 snug region in combination with a relatively small inner diameter of the lumen 368 of the catheter advancement element 300, typically filled by the guidewire and/or liquid, results in the catheter advancement element 300 being relatively occlusive to the aspiration catheter 200 when positioned in its lumen 223 and forming a closed system. The catheter advancement element 300 is generally removed before any external aspiration is applied through the lumen 223 of the aspiration catheter 200. For example, the catheter advancement element 300 can be positioned at its distal-most position and the aspiration catheter 200 can be advanced to a location, the catheter advancement element 300 can be withdrawn proximally back into the lumen 223 of the aspiration catheter 200. The snug region between the two tubes also moves proximally

### Venous Sinus Stents and Delivery Systems

Conventional intravascular stents and delivery systems have been used to treat venous sinus stenoses. Both the anatomy to reach the venous sinuses and the venous sinuses themselves differ in important ways to other clinical applications for which conventional stents have been developed. Intravascular stents are conventionally designed for treating vascular blockages in vessels that are primarily circular in cross-section. The venous sinuses are not circular nor are they compliant. In contrast to blood vessels formed from vascular wall layers including a muscular tunica media, the venous sinus lumens are formed from channels between layers of the dura mater, a tough non-compliant membrane surrounding the brain. Advancing catheters, especially larger access and treatment devices, through these rigid and tortuous channels is difficult with current access systems. Deploying standard circular implants into these non-round and non-conformal channels leads to sub-optimal results. There are opportunities to improve the speed and safety of stent implantation, as well as long term improvement of results of the stent itself.

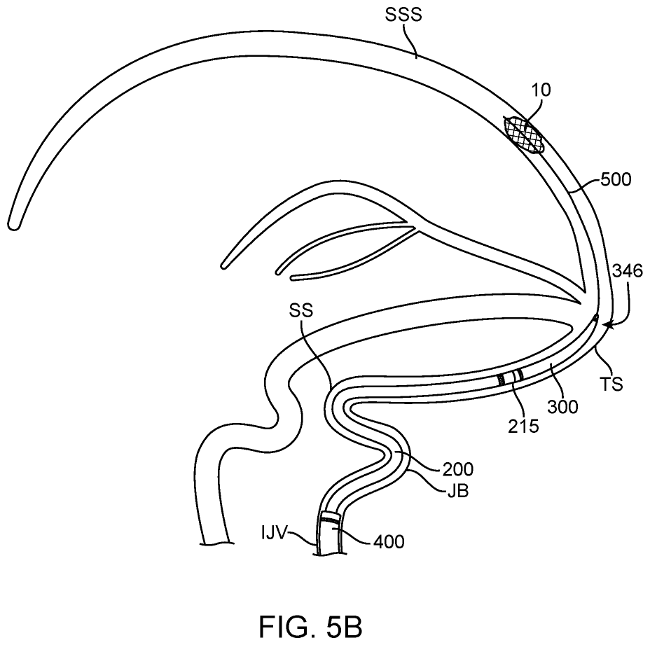

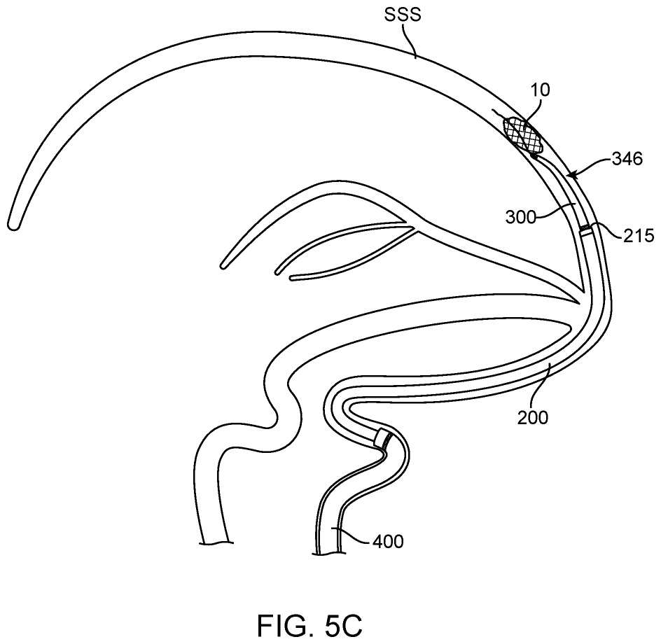

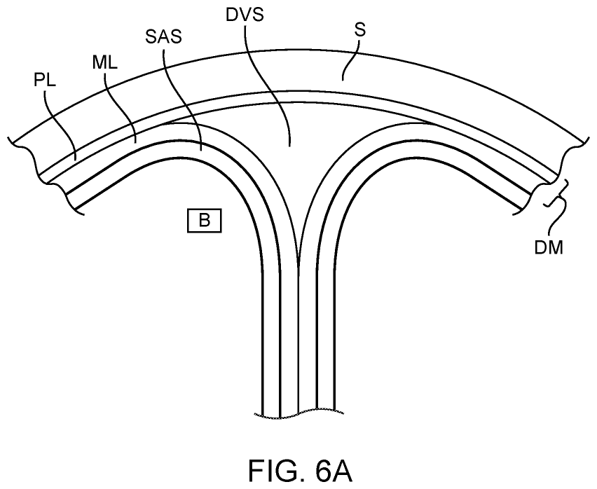

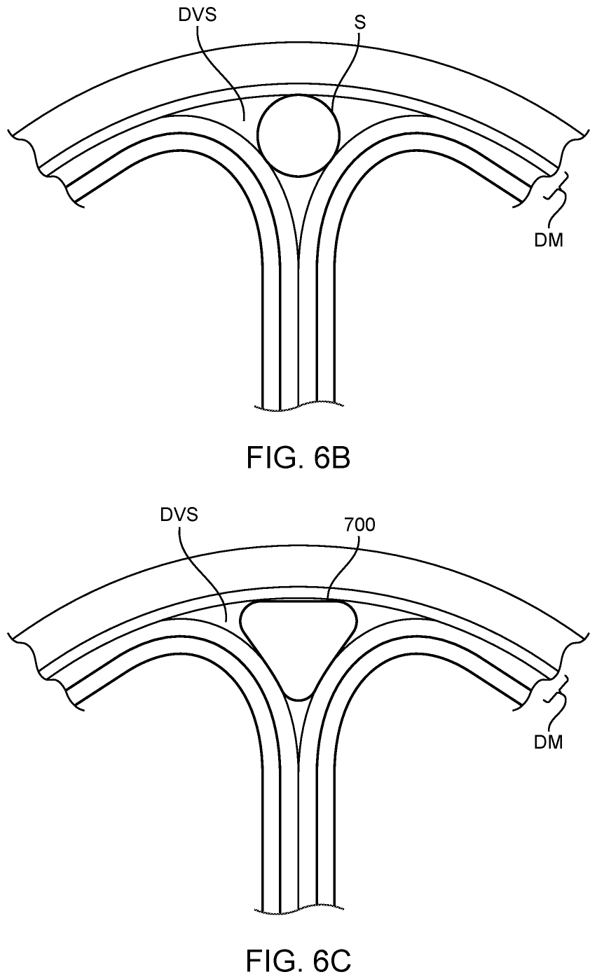

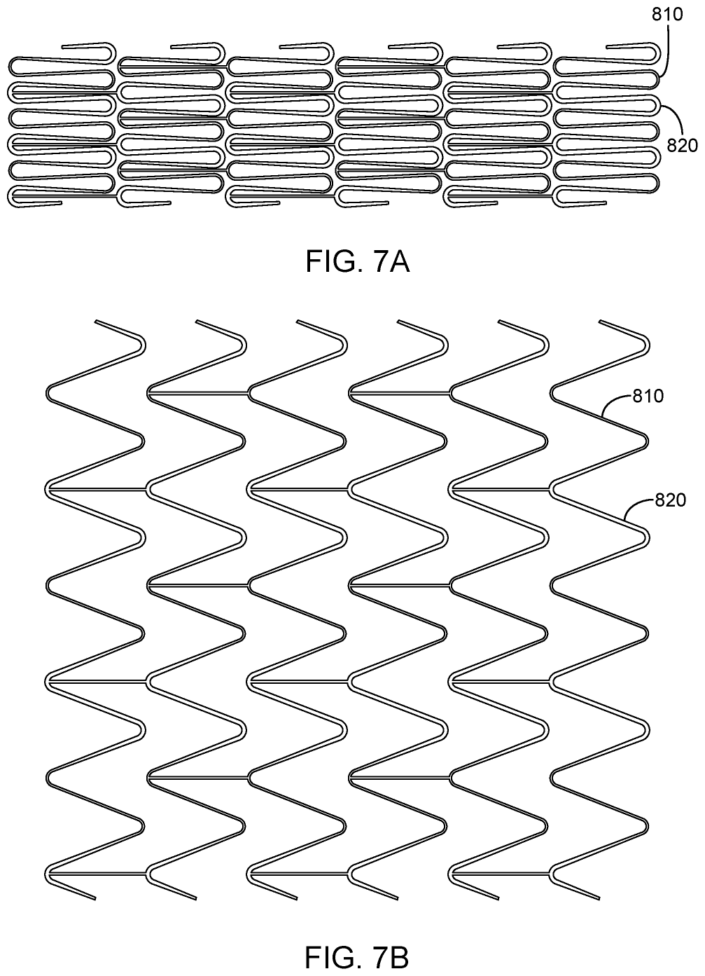

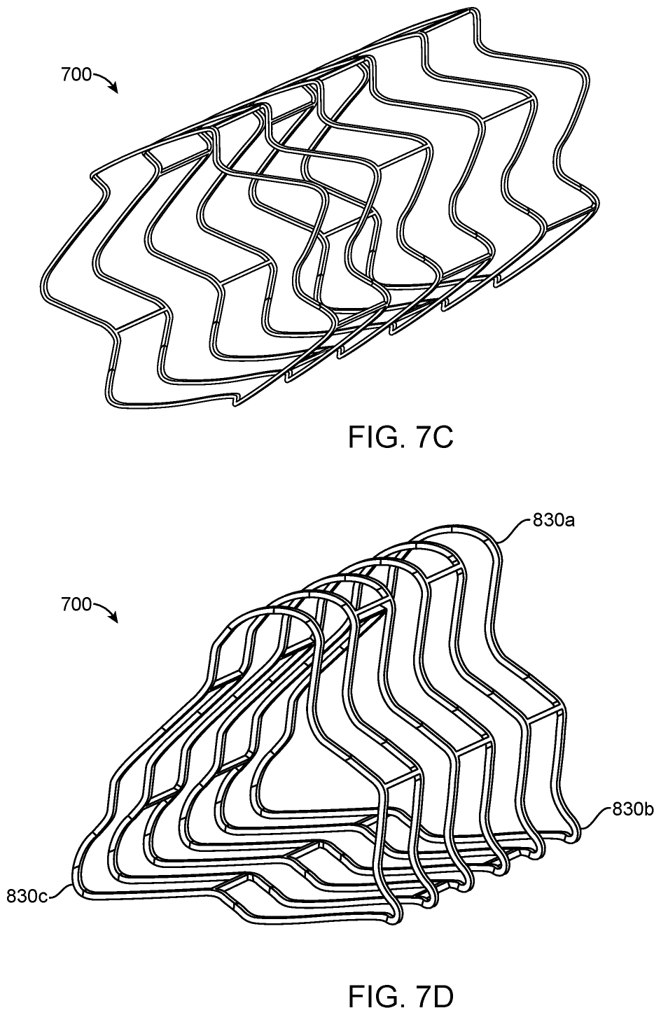

FIG. 6A shows the structure of the venous sinus luminal space and surrounding anatomy. The dura mater DM is a thick and tough structure surrounding the brain and located on the inner surface of the skull S and includes two layers, the periosteal layer PL adjacent to the skull S, and the meningeal layer ML beneath the periosteal layer PL. The two layers are for the most part in contact with each other. Beneath the dura matter DM is the sub-arachnoid space SAS, which is filled with cerebral spinal fluid and acts as a cushion to protect the brain B. In specific areas of the dura mater DM, for example, locations where the meningeal layers fold to form barrier structures within the brain sections, the periosteal layer PL and meningeal layer ML separate to form channels, which are the dural venous sinuses DVS. The dural venous sinuses DVS form the drainage from the cerebral veins and the skull veins (not shown) to the internal jugular veins, and from there back to the heart. The periosteal layer PL and the meningeal layer ML are relatively tough, and the sinus channels DVS between them do not form a circular or continuous lumen. Therefore, conventional stents, which have been implanted here to treat stenoses, do not have good apposition to the luminal walls, which in turn may lead to stasis areas that result in thrombotic occlusion of the stents, and/or may not provide optimal opening of the stenotic material in the venous sinus. FIG. 6B shows a traditional stent S having a circular shape positioned within the non-circular channel of the dural venous sinus DVS. FIG. 6C shows a stent 700, such as the stents illustrated in FIGS. 7A-7D, designed specifically for a venous sinus having a non-circular shape positioned within the non-circular channel of the dural venous sinus DVS.

FIGS. 7A-7B are unrolled, flat views of an implementation of an implantable device that is a venous sinus stent 700 in an unexpanded and expanded configuration, respectively. The implantable device includes a tubular member defining a lumen having a longitudinal axis from a proximal end to a distal end of the tubular member. The tubular member is configured to transition from an unexpanded configuration towards a fully expanded, deployed configuration. The unexpanded configuration can have a first cross-sectional shape (the cross-section taken perpendicular to the longitudinal axis of the lumen) that is substantially round without sides. The deployed configuration can have a second cross-sectional shape (the cross-section taken perpendicular to the longitudinal axis of the lumen) that is substantially non-round and multi-sides. FIGS. 7A-7B show a flat pattern of an implementation of a venous sinus stent 700 in a compressed or non-expanded state or unexpanded configuration and an expanded state or deployed configuration, respectively. The stent 700 is designed to adapt to the non-conformal and generally triangular cross-sectional lumen of the dural venous sinuses DVS. In one implementation, the stent 700 is designed to be formed in situ after deployment from the first cross-sectional shape to the second cross-sectional shape, such as with a balloon. For example, the stent 700 can be deployed from the delivery system to a first expanded configuration having a first cross-sectional shape, such as a substantially round shape in the first expanded configuration. The stent 700 can then be formed into a second expanded configuration having a second cross-sectional shape that is different from the first cross-sectional shape. For example, the stent 700 can be expanded or formed in situ to change from the substantially round shape shown in FIG. 7C into a non-round shape in the second expanded configuration, such as a three-sided triangular shape shown in FIG. 7D. The second expanded configuration can have any of a variety of shapes including round, elliptical, oval, square, triangular, or other geometric shape that is different from the first expanded configuration. The second expanded configuration can be the same shape as the first expanded configuration, but have a larger diameter than the first expanded configuration. The second expanded configuration can be the same shape as the first expanded configuration, but can be a distortion of the same shape. The shape of the second expanded configuration can include rounded corners and generally flat sides so that the shape of the second expanded configuration is more multi-sided than round whereas the first expanded configuration is more round than multi-sided.

The first cross-sectional shape can be circular, oval, or elliptical and the second cross-sectional shape can be square or triangular. The second cross-sectional shape can have rounded corners and generally flat sides. The rounded corners can have a tighter bend radius upon expansion of the tubular member and can be formed by thinner, more malleable struts than struts forming the generally flat sides formed by thicker, less malleable struts. The tubular member can be further configured to transition from the unexpanded configuration to a transitional configuration before transitioning into the deployed configuration. The transitional configuration can have a transitional cross-sectional shape (cross-section taken perpendicular to the longitudinal axis of the lumen) that is substantially round without sides. The transitional cross-sectional shape can be the same shape or a distorted version of the first cross-sectional shape that has a transitional diameter that is larger than a diameter across the first cross-sectional shape. The first cross-sectional shape is configured to be inserted through the vasculature whereas the second cross-sectional shape is configured to mate with luminal dimensions of a venous sinus.

The transitional configuration need not be round, but can be a similar shape as the second cross-sectional shape, but distorted or smaller in overall diameter. Thus, the first unexpanded configuration can have a first cross-sectional shape such as circular, oval, or elliptical, the transitional configuration can have a second cross-sectional shape such as triangular or square, and the second expanded configuration can have a third cross-sectional shape such as triangular or square that is expanded and has a larger overall area than the second cross-sectional shape. In practice, the stent 700 can be delivered to a treatment site such as a venous sinus while in the first unexpanded configuration and the outer sleeve retracted so that the stent 700 expands to the transitional configuration having a non-round cross-sectional shape, such as triangular. Once in the transitional configuration, the user can manipulate the orientation of the tubular member so that the corners and sides of the tubular member in the transitional configuration can be aligned to mate with the corresponding anatomy once fully expanded to the deployed configuration. For example, the tubular member can be torqued in one direction or another around the longitudinal axis of the lumen of the tubular member to better align the corners of the tubular member with corresponding corners of the target venous sinus and to align the substantially flat sides of the tubular member with the corresponding sides of the venous sinus. Once oriented as desired in the target venous sinus, the stent 700 can then be fully expanded to the second, expanded configuration so that the flat sides appropriately engage with the walls of the venous sinus without inadvertently placing a corner of the tubular member where a flat side should be located. Thus, the stent is configured to be partially expanded to allow for orienting the stent within the target anatomy so that the non-circular shape of the stent can mate with the non-circular shape of the venous sinus prior to full expansion and engagement with the walls of the sinus. The reorienting of the partially expanded stent can be performed using the delivery system, which will be described in more detail below.

Again with respect to FIGS. 7A-7B, the stent pattern can be repeated by a number divisible by 3, for example 6, 9, 12, etc. The flat pattern shows a repeating pattern of 6 around the circumference. The stent 700 can be made more bendable in various sections along the longitudinal axis by varying the thickness of the struts. For example, three sets of struts around the circumference can be thinner, such as strut 810, and the other three sets of struts can be thicker, such as strut 820, so as to make the stent 700 more bendable in three sections along the longitudinal axis. The stent can incorporate more than 6 strut sets around the circumference. In this example, three sets of struts can be thinner like strut 810 and the remainder would be thicker.

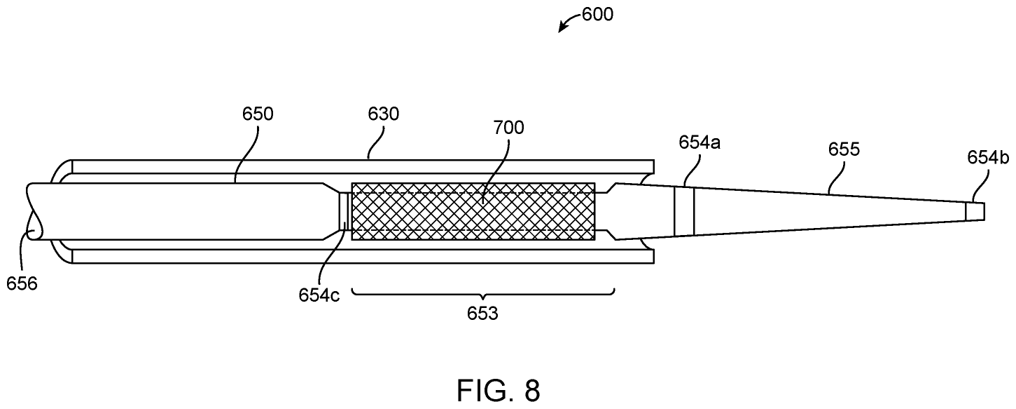

FIG. 8 illustrates a distal section of venous sinus stent and delivery system 600. The delivery system 600 can include an inner member 650 and an outer member or outer sleeve 630. A tubular member formed by a plurality of struts that define a lumen having a longitudinal axis from a proximal end to a distal end of the tubular member can be mounted on the inner member and constrained by the outer member during delivery. The inner member 650 can be an elongate structure having a recessed area 653 located a distance away from the distal-most end. The recessed area 653 can have an outer diameter that is less than an outer diameter of the inner member 650 proximal to the recessed area 653. The outer diameter of the recessed area 653 is sized to receive the venous sinus stent 700 in the compressed configuration so that the venous sinus stent 700 can be received within the recessed area 653 of inner member 650 and still be covered by the outer sleeve 630 in a manner that allows for relative movement between the outer sleeve 630 and the inner member 650. The recessed area 653 can be recessed relative to the outer diameter of the inner member 650 by a thickness of the stent 700 in the compressed configuration so that the overall outer diameter of the inner member 650 is substantially uniform when the stent 700 is received within the recessed area 653.

The outer sleeve 630 of the stent delivery system 600 is configured to keep the stent 700 constrained in recessed area 653 of inner member 650, but once at the target site may be retracted to deploy stent 700. The outer sleeve 630 can be retracted relative to the inner member 650 by a distance sufficient to expose the stent 700 positioned within the recessed area 653 for deployment at the treatment site. The recessed area 653 is sufficiently recessed so that the stent 700 can be received within the recessed area 653 and the outer sleeve 630 easily retracted over the top of the stent 700 for deployment. The outer sleeve 630 may include a lubricious inner liner, such as a PTFE or FEP inner liner. The outer member may also include longitudinal elements, such as a fiber or wire embedded into the wall, to prevent stretching when the outer sleeve 630 is retracted to deploy the stent 700. The tubular member is configured to transition from an unexpanded configuration towards a fully expanded, deployed configuration as described above where the unexpanded configuration has a first cross-sectional shape (taken perpendicular to the longitudinal axis of the lumen) that is substantially round without sides and the deployed configuration can have a second cross-sectional shape (taken perpendicular to the longitudinal axis of the lumen) that is substantially non-round and multi-sided. The first cross-sectional shape can be circular, oval, or elliptical whereas the second cross-sectional shape can be square or triangular. The tubular member can be configured to transition from the unexpanded configuration to a transitional configuration as discussed above before transitioning into the deployed configuration that is shaped to mate with the luminal dimensions of a venous sinus.

Both the inner member 650 and outer sleeve 630 have a construction that, once assembled together and with the venous stent 700 to form system 600, has adequate flexibility and pushability be able to be advanced in a relatively rapid and atraumatic fashion to the desired venous sinus treatment site.

The inner member 650 of the delivery system 600 can aid in navigating the delivery system 600 through anatomy leading to the venous sinus similar to the catheter advancement element 300 described above. The venous sinuses have irregular walls including fibrous bands called chordae willisii stretching transversely across the sinus, and small openings that communicate with irregularly shaped venous spaces and serve as the drainage channels into the venous sinuses, making smooth advancement of devices difficult and sometimes dangerous. The inner member 650 has an atraumatic distal end region 655 so as to minimize vessel trauma during advancement of delivery system 600. In an embodiment, the distal end region 655 can be tapered with design features similar to distal tapered tip 346 of catheter advancement element 300.

Radiopaque markers 654a and 654b delineate the tapered section of the distal end region 655 for the user under fluoroscopy. As noted previously, the tapered distal end region 655 in addition to providing atraumatic advancement through extreme tortuosity can also alert the user to advancement in a false lumen, as illustrated in FIG. 2C and described previously with reference to the tapered tip element 346 of catheter advancement element 300. The recessed area 653 can also incorporate one or more radiopaque markers 654c so that the location of the stent 700 received within the recessed area 653 can be identified during use of the delivery system 600. The recessed area 653 can include one or more features that aid in retaining the stent 700 within the recessed area 653.

The inner member 650 can have variable flexibility formed by a plurality of shaft portions made of different materials to increase flexibility from the proximal end region towards the distal tip. The proximal end region can incorporate a hypotube or other stiffening element to provide some pushability to the inner member 650 as discussed elsewhere herein. The distal-most end of the distal end region 655 can be formed of a medical grade polymer such as PEBAX 35D. The durometer of the regions between the distal-most end and the proximal end region can vary with increasing stiffness further away from the distal end of the inner member 650.

The inner member 650, when used with a self-expanding stent, may also include a single lumen 656 sized to accommodate a guidewire, to enable advancement of the system 600 over a guidewire. In an embodiment, the lumen 656 is configured to accommodate a 0.014″ or 0.018″ guidewire. Specifically, the lumen inner diameter is in the range of 0.015″-0.024″, or preferably no greater than about 0.022″, no greater than about 0.020″, or no greater than about 0.018″.

The stent 700 can be a balloon-expandable stent or self-expanding. As such, the delivery system 600 can be configured for deployment of a balloon-expandable stent or a self-expanding stent. For example, with a self-expanding stent, the outer sleeve 630 can constrain the stent 700 during delivery. However, for a fully balloon-expandable portion, the stent delivery system 600 may not incorporate the outer sleeve 630 to keep the stent mounted on the delivery system 600. Similarly, for a balloon-expandable stent, the inner member 650 may include the guidewire lumen 656 as well as an additional inflation lumen (not shown) for expanding the balloon at a distal end region of the inner member 650.

The stent 700 can be crimped onto the recessed area 653 of the inner member 650, which may have a distal balloon as discussed above, and delivered to the treatment site. Once positioned, the balloon can be inflated to deploy the stent 700 at the treatment site into its first expanded configuration. Alternatively, the outer sleeve 630 can be retracted to deploy the stent 700 such as by self-expansion. The stent 700 can deploy into a first expanded configuration taking on a generally rounded cross sectional shape as shown in FIG. 7C. The stent 700 can then be post-dilated with a balloon (the same balloon used to initial deploy the stent or a separate balloon) into its second expanded configuration. The second expanded configuration can be a generally non-round cross-sectional shape having a plurality of sides and a plurality of corners, as shown in FIG. 7D. As discussed above, the non-round cross-sectional shape can vary including elliptical, oval, square, triangular, or other geometric shape. The distal balloon used to post-dilate the stent 700 can be balloon shaped to have the non-round cross-sectional shape or a conformal balloon that takes the shape of the dural venous sinus lumen and thereby forces the stent 700 against the walls of the dural venous sinus DVS.

In an alternate embodiment, the stent 700 has sections with different material characteristics that provide for initial deployment into a first shape and post-deployment into a second, different shape. For example, the stent 700 can have a first section(s) that is malleable and a second section(s) that is springy. The second springy sections allow the stent 700 to be deployed into a first shape (e.g., round) when the outer sleeve 630 is retracted. The malleable section(s) allow the stent 700 to be further deformed in situ into a second shape (e.g., non-round), for example, with a conformal or generally triangular-shaped balloon. Again referring to FIGS. 7A and 7B, the thinner struts 810 may also be heat formed to be more malleable than thicker struts 820 so that the thinner struts 810 form a weakened area within the circumference of the stent that would bend more easily than areas formed by the thicker struts 820. The thicker struts 820 provide a springiness to the stent 700 such that the stent can be restrained by the outer sleeve 630 to a compression shape suitable for delivery through a vessel and then expand upon withdrawal of the outer sleeve 630 to an initial expanded shape. The thinner struts 810 that are malleable for a user to form the stent 700 into a second expanded shape that is different from the initial expanded shape such as by increasing a radius of curvature of the stent 700 at some locations while decreasing a radius of curvature of the stent 700 at other locations to create flat sides that meet at rounded corners 830a, 830b, 830c. The rounded corners 830a, 830b, 830c having a tighter bend radius can be formed by the thinner struts 810 that bend more easily and the flatter sides can be formed by the thicker struts 820 that bend less easily.

In another embodiment, the stent is heat formed during manufacturing into a three-sided, generally triangular shape. The three-sided cross-sectional shape conforms better to the shape of the dural venus sinus, as shown in FIG. 6C, assuming the sides of the stent 700 (rather than the rounded corners 830a, 830b, 830c of the stent 700) are positioned against flatter surfaces of the sinus. In order to confirm that this non-round shape is oriented appropriately in the non-round lumen (i.e., so that the rounded corners 830a, 830b, 830c of the stent 700 align generally with corners of the sinus), the stent delivery system 600 can have means to orient itself in the non-round lumen of the sinus. For example, the stent delivery system 600 may have protruding features or a distal triangular balloon that will “self-orient” into the triangular lumen. The protruding features can be on the inner member 650 or the sleeve 630.

The stent can be a balloon-expandable stent and malleable, formed from martensitic nitinol, stainless steel SS, cobalt chromium CoCr or other metals and metal alloys known for use in balloon-expandable stents. The stent can be a self-expanding stent similar in design to carotid stent systems, such as the WALLSTENT (Boston Scientific), PRECISE Stent (Cordis), ZILVER (Cook), and others. Alternately, the stent can be designed to accommodate the differentiated luminal space of the dural venous sinuses.

### Methods

The catheter systems described herein can be used to access and treat a dural venous sinus occlusion, which can be a superior sagittal sinus, by removing thrombotic material with aspiration and/or by delivering a stent such as the stent 700 described above and as shown in FIGS. 7A-7B, and 7C-7D.

The patient can be treated initially with anticoagulants or fibrinolytic agents like recombinant tissue plasminogen activator (rtPA), or anti-platelet therapy, such as heparin. A base sheath 400 can be inserted into a vessel from an access site, such as the femoral vein in the groin of the patient, and advanced at least to a level of the internal jugular vein. The base sheath 400 may be placed on continuous flush with an RHV. The base sheath 400 can be advanced over a guidewire. The base sheath 400 can be positioned to sites distal to the internal jugular vein (“distal” here in relation to the access site location as opposed to natural flow of blood through the vein) depending on which dural venous sinus is being treated. For example, treatments in the superior sagittal sinus may be improved by advancing the distal end of the base sheath 400 to a region of the transverse sinus. The catheter 200 (which may be preloaded with a matching catheter advancement element 300) is inserted into the base sheath 400. The catheter 200 and catheter advancement element 300 can be inserted as a unit or individually in nested fashion. The catheter 200 may be an 0.070″ (1.778 mm), preferably an 0.088″ (2.235 mm) or larger catheter having a suitably sized catheter advancement element 300 positioned within its lumen so that a snug point between the two components is created. A procedural guidewire 500 can be positioned within the lumen of the catheter advancement element 300, which is positioned within the lumen of the catheter 200. The assembled system 150 can be inserted through the proximal hemostasis valve 434 of the guide sheath 400.

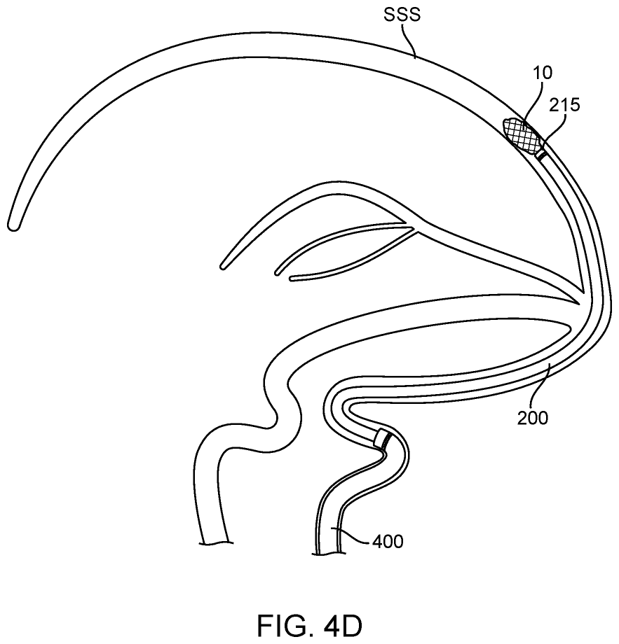

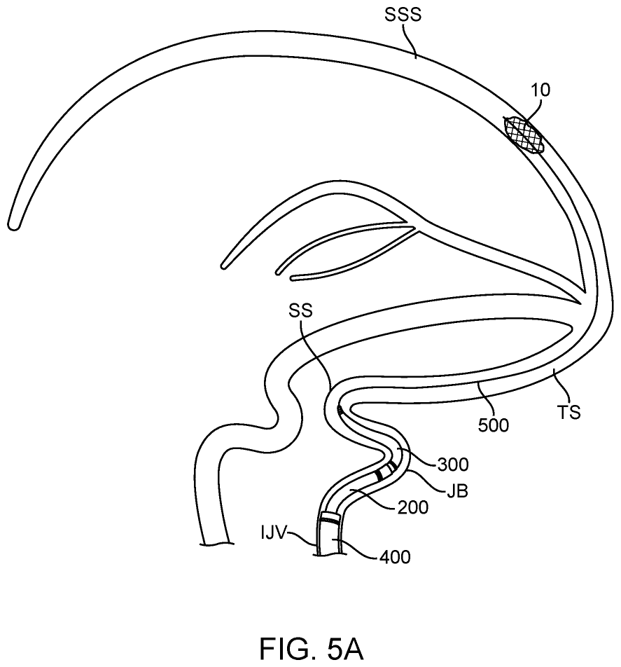

FIGS. 4A-4D illustrate a method of advancement of the catheter system towards an occlusion in the superior sigmoid sinus SSS without a guidewire or with a guidewire parked within the lumen of the catheter advancement element. FIGS. 5A-5C illustrate an alternative method of advancement of the catheter system over a guidewire positioned distal to the occlusion. The jugular bulb JB is a dilation of the upper bulbous portion of the jugular vein IJV. A first turn occurs between the jugular bulb JB and the sigmoid sinus SS. A second turn occurs between the sigmoid sinus SS and the transverse sinus TS. The guidewire 500 can be advanced first ahead of the catheter system 150 to navigate the turns off the jugular bulb JB. The guidewire 500 can be advanced into the jugular bulb JB to select the sigmoid sinus SS. The catheter advancement element 300 can be advanced over the guidewire 500 to create a smooth curve along the top of the bulb JB into the sigmoid sinus SS. The catheter 200 can be advanced over the catheter advancement element 300 so that the distal end 215 of the catheter 200 is directed towards or is positioned within the sigmoid sinus SS without kinking and without formation of a distal lip. The guidewire 500 can then be advanced into to select the transverse sinus TS and the catheter advancement element 300 advanced over the guidewire 500 to create a smooth curve into the transverse sinus TS. The catheter 200 can be advanced over the catheter advancement element 300 so that the distal end 215 of the catheter 200 is directed into the transverse sinus TS without kinking and without formation of a distal lip. Alternatively, the catheter 200 having the catheter advancement element 300 extending distal to the distal end 215 of the catheter 200 can be advanced together over the guidewire 500 into the sigmoid sinus SS and transverse sinuses TS. The guidewire 500 can be advanced up to the level of the occlusion 10 or across to the distal side of the occlusion 10 as shown in FIGS. 5A-5C. Further alternatively, the catheter 200 and the catheter advancement element 300 can be advanced through the jugular bulb JB into the sigmoid sinus SS and further into the transverse sinus TS without a leading guidewire 500 as illustrated in FIGS. 4A-4D. The guidewire 500 can be parked within the lumen of the catheter advancement element 300 as the components are advanced simultaneously through the turns. Once the assembled system 150 is advanced to the level of the transverse sinus TS, it can be used as a support for advancing the guide sheath 400 further distal, if desired. The transverse sinus TS tends to have more tributaries and septations that render navigation with a guidewire through the transverse sinus TS particularly challenging and prone to risk. To navigate through the transverse sinus TS to the superior sagittal sinus SSS, the guidewire 500 is positioned inside the lumen of the catheter advancement element 300 so that the catheter advancement element 300 leads the catheter system 150 during navigation. The catheter advancement element 300 can navigate through the confluence into the superior sagittal sinus SSS towards the occlusion 10.

The catheter advancement element 300 can be advanced until the distal tip region 346 probes the proximal face of thrombotic occlusion 10 as shown in FIG. 4C, where “proximal” is in relation to the advancement direction with proximal being nearer the initial access site and “distal” being further away from the initial access site. Advancing the catheter advancement element 300 can include positioning the distal end 325 of the catheter advancement element 300 between a portion of the thrombotic occlusion 10 and the sinus wall. Advancing the catheter advancement element 300 can include positioning the distal end 325 of the catheter advancement element 300 without crossing the thrombotic occlusion 10 with the distal end 325 of the catheter advancement element 300. Advancing the catheter advancement element 300 can include positioning the distal end 325 of the catheter advancement element 300 as far as possible without buckling of the catheter advancement element 300. Advancing the catheter advancement element 300 can include interrogating the treatment site to locate the proximal face of the thrombotic occlusion Advancing the catheter advancement element 300 can include using the tapered distal end region 346 of the catheter advancement element 300 to dissect past the soft clot material to probe for denser material. The thrombotic occlusion 10 may or may not have denser embolus or firmer fibrous clot. Typically, thrombotic occlusion in the venous sinuses is relatively fresh and less organized. Veins are relatively flexible compared to arteries so that as the devices are advanced through them, the thrombotic material (whether organized or not) tends to move out of the way as the vein stretches.

The catheter 200 can be advanced over the catheter advancement element 300 to position the distal end 215 of the catheter 200 at a treatment site located sufficiently close to the thrombotic occlusion to capture thrombotic material within the catheter 200 as shown in FIG. 4D. The catheter advancement element 300 can be removed from the lumen of the catheter 200. Aspiration can be applied to the catheter 200.

The catheter 200 is preferably size-matched to the inner diameter of the venous sinus or as large as possible to navigate to the venous sinus being treated so that the catheter distal end 215 can be advanced to partially engulf or surround a portion of the thrombotic occlusion. The distal end of the catheter 200 can be placed close to the thrombotic occlusion for efficient aspiration. An outer diameter of the catheter 200 can be sized close to the venous sinus size to maximize efficiency of aspiration. Thus, for an occlusion in the superior sagittal sinus the catheter 200 may be no smaller than 0.088″ inner diameter up to about 0.130″ inner diameter and in some anatomy as large as 0.160″. An 0.088″ catheter would be significantly smaller than the superior sagittal sinus in the occipital segment, but becomes size-matched moving forward into the frontal segment. The catheter can be larger than 0.088″, including to about 0.160″ depending on the target anatomy to be treated. The size of the access vessel may determine the maximum size of the catheter that can be advanced (as opposed to the size of the target vessel to be treated). For example, the common femoral vein used as an access site may vary widely in size and in some patients can be as big as 10 mm, but generally is large enough to receive an 0.088″ catheter.

The method can include one or more steps of injecting contrast agent to assess the occlusion site by angiogram. The method can also include one or more steps of advancing and re-advancing the catheter system 150 to the occlusion site to continue removing thrombotic material from the occlusion site until outflow through the sinus is confirmed. The occlusive material can be captured at, within, or through the distal end 215 of the catheter 200 while applying aspiration from an external aspiration source, for example, an aspiration source coupled to the RHV 434 of the base sheath 400. The method can additionally or alternatively include one or more steps of mechanical embolectomy using a retrievable structure, such as a coil-tipped retrievable stent, a woven wire stent, or a laser cut stent, or other expandable device configured to engage the clot and remove thrombotic material through the catheter with or without aspiration. The retrievable device can be a stent retriever, such as SOLITAIRE (Medtronic) or TREVO (Stryker) or other device known in the art.

A related method of using the catheter systems described herein includes accessing and treating a dural venous sinus, such as the sigmoidal, transverse, or superior sagittal sinus, by stenting an occlusion or stenosis with a deployable expandable device, either a conventional vascular stent with the appropriate diameter and length dimensions and mechanical properties, or the venous sinus stent disclosed herein and as shown in FIGS. 7A-7B, and 7C-7D. The patient can be treated initially with systemic or local anti-coagulants as described above. If thrombolytic therapy is unsuccessful, direct catheter thrombolysis can be performed. Direct catheter delivery of a thrombolyic agent involves a microcatheter and guidewire delivered to the thrombosed dural sinus through a sheath or guiding catheter from the jugular bulb. Mechanical manipulation of the thrombus with the guidewire increases the amount of clot that might be impacted by the thrombolytic agent as it is delivered through the microcatheter. A base sheath 400 can be inserted into a vessel from an access site, such as the femoral vein in the groin of the patient, and advanced through the venous system to at least the internal jugular vein. The base sheath 400 may be placed on continuous flush with an RHV. The base sheath 400 can be advanced over a guidewire. The base sheath 400 can be positioned to sites distal to the internal jugular vein depending on which dural venous sinus is being treated as discussed above. If an additional catheter is needed to reach the target site in the dural sinus, the catheter 200 (which may be preloaded with a matching catheter advancement element 300) is inserted into the base sheath 400. The catheter 200 and catheter advancement element 300 can be inserted as a unit or individually in nested fashion. A procedural guidewire 500 can be positioned within the lumen of the catheter advancement element 300, which is positioned within the lumen of the catheter 200. Upon reaching a target treatment location, the advancement element 300 is removed from the lumen of the catheter, and the stent delivery catheter 600 can be inserted through the proximal hemostasis valve 434 of the guide sheath 400 into the catheter 200. Alternately, the stent delivery catheter 600 is directly inserted into base sheath 400 and advanced over a guidewire into the dural sinus region.

The stent delivery system 600 can be advanced into the sigmoidal or transverse sinus as described above. If the treatment area is one of these sites, the stent can be deployed at this time. If the site is distal to the transverse region, navigation to distal regions can be performed with or without a guidewire leading the system 600. For example, the guidewire can be parked inside the lumen of the inner member 650 of the stent delivery system 600 and the stent delivery system 600 can navigate to the treatment site. Alternately, the guidewire may be advanced distal to the target region and the delivery system 600 can be advanced to the more distal target site, for example the superior sagittal sinus, over the guidewire. Once the delivery system 600 is at the treatment site, the stent is deployed, for example by retracting outer member 630. If the expanded stent has a circular expanded cross-sectional shape, the stent would sit in the dural venous sinus DVS as shown in FIG. 6B. The expanded stent can be formed in situ from the circular expanded cross-sectional shape into a non-circular expanded cross-sectional shape, such as a three-sided shape, configured to conform to the shape of the dural venous sinus DVS, as described in more detail above. If desired, the stent delivery system 600 can be exchanged for a conformal or triangular-shaped balloon, and the balloon can be inflated to form the stent 700 from a round shape, as shown in FIG. 7C, into the non-round, generally triangular shape of the dural sinus, as shown in FIG. 7D and FIG. 6C.

Alternately, if the stent is pre-formed into a generally triangular shape as shown in FIG. 7D, the stent delivery system 600 can be oriented in the correct radial orientation to deploy the stent in the desired radial orientation in the dural sinus lumen. For example, the stent delivery system 600 can be manipulated such that the protruding features are located in the three corners of the generally triangular lumen of the dural sinus. The triangular stent 700 can then be deployed in the correct orientation in the lumen, as shown in FIG. 6C. If desired, the triangular stent 700 can be post-dilated to further optimize apposition of the stent 700 against the generally triangular lumen of the dural sinus.

Another related method of using the catheter systems described herein includes accessing a dural venous sinus, such as the superior sagittal sinus, to deliver an implant, such as an electrode, for stimulating the brain for the purposes of treating a neurological condition. The catheter advancement element 300 can be advanced until the distal tip region 346 probes the occlusion. Advancing the catheter advancement element 300 can include positioning the distal end 325 of the catheter advancement element 300 as far as possible without buckling of the catheter advancement element 300, which can be detected by a combination of feel and fluoroscopy. The catheter 200 can be advanced over the catheter advancement element 300 to position the distal end 215 of the catheter 200 near the occlusion as described in U.S. application Ser. No. 18/311,797, filed May 3, 2023, which is incorporated by reference herein. The catheter advancement element 300 can be removed from the lumen of the catheter 200. The catheter 200 can be remain in place and the stent delivery system 600 advanced through the catheter 200 to the site of the occlusion and used to deploy the stent at the site of the occlusion. S tents delivered at the site of occlusion can be up to about 10 mm in diameter and very long lengths. The stents can be tapered or straight, preferably with anti-thrombogenic coating. The stent can be a metallic open cells stent having minimal metal or can be resorbable. The method can include one or more steps of injecting contrast agent to assess the occlusion site by angiogram as well as applying aspiration from an external aspiration source, for example, coupled to the RHV 434 of the base sheath 400 during one or more steps of the method.

The catheter 200 may be an 0.070″ (1.778 mm), preferably an 0.088″ (2.235 mm) or larger catheter having a sized-matched catheter advancement element 300 positioned within its lumen so that a snug point between the two components is created. The size of the catheter 200 and its size-matched catheter advancement element 300 can be selected to accommodate the outer diameter of the stent delivery system, which is selected to deploy a stent having a size suitable for the venous sinus being treated. The catheter 200 can support the deployment of carotid stents, such as WALLS TENT (Boston Scientific) or PRECISE Stent (Cordis), ZILVER (Cook), and others. Alternately, the catheter 200 can support the advancement and deployment of a venous sinus stent 700 and delivery system 600. The stent can be between about 6 mm up to about 10 mm in diameter depending on the sinus being treated and can vary in length. The smaller sized stents can be delivered through a catheter 200 that is about 0.070″ inner diameter whereas the larger sized stents are delivered through larger inner diameters of about 0.088″. Because of the thromboembolic risks associated with longer stents, particularly in the cerebral vessels, shorter stents or stents having a length that substantially matches the length of the stenosis or occlusion being treated is preferred. The stent deployed can be self-expanding or balloon-expanded. The stent deployed can be a resorbable stent or a permanent stent.

### Materials

One or more components of the catheters described herein may include or be made from a variety of materials including one or more of a metal, metal alloy, polymer, a metal-polymer composite, ceramics, hydrophilic polymers, polyacrylamide, polyethers, polyamides, polyethylenes, polyurethanes, copolymers thereof, polyvinyl chloride (PVC), PEO, PEO-impregnated polyurethanes such as Hydrothane, Tecophilic polyurethane, Tecothane, PEO soft segmented polyurethane blended with Tecoflex, thermoplastic starch, PVP, and combinations thereof, and the like, or other suitable materials.

Some examples of suitable metals and metal alloys for catheter braid or coil reinforcement include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material and as described elsewhere herein.

Inner liner materials of the catheters described herein can include low friction polymers such as PTI-E, (polytetrafluoroethylene) or FEP (fluorinated ethylene propylene), PTFE with polyurethane layer (Tecoflex). Reinforcement layer materials of the catheters described herein can be incorporated to provide mechanical integrity for applying torque and/or to prevent flattening or kinking such as metals including stainless steel, Nitinol, Nitinol braid, helical ribbon, helical wire, cut stainless steel, or the like, or stiff polymers such as PEEK. Reinforcement fiber materials of the catheters described herein can include various high tenacity polymers like Kevlar, polyester, meta-para-aramide, PEEK, single fiber, multi-fiber bundles, high tensile strength polymers, metals, or alloys, and the like. Outer jacket materials of the catheters described herein can provide mechanical integrity and can be contracted of a variety of materials such as polyethylene, polyurethane, PEBAX, nylon, Tecothane, and the like. Other coating materials of the catheters described herein include paralene, Teflon, silicone, polyimide-polytetrafluoroetheylene, and the like. The inner liner may further include different surface finishes, such as dimples, bumps, ridges, troughs. The surface finishes may be randomly disposed, linearly disposed, spirally disposed, or otherwise disposed using a specific pattern along the length of the catheter. It is further contemplated that the inner liner may include a mixture of different surface finishes, for example, one section may have dimples, another section may have troughs, etc. Additionally, the surface finish may be incorporated along the entire length of the catheter or only in sections of the catheter. It is also contemplated that the inner liner may further include an electrosprayed layer, whereby materials could be incorporated into the inner liner. Examples of materials can include low friction materials as described above. Alternatively, the electrosprayed or electrospun layer may incorporate a beneficial agent that becomes free from the coating when exposed to blood, or to compression from a clot, for example, the beneficial agent may be a tissue plasminogen activator (tPA) or heparin encased in alginate.

Materials for venous sinus implants include metals used for endovascular implant devices, such as Nitinol, 316 stainless steel, such as 316SS, cobalt chromium or other cobalt alloys, elgiloy. Metals used for radiopaque marker materials include gold and gold alloys, platinum, platinum-iridium and other platinum alloys, tantalum and tantalum alloys.

Implementations describe catheters and delivery systems and methods to deliver catheters to target anatomies. However, while some implementations are described with specific regard to delivering catheters to a target vessel of a neurovascular anatomy, such as a dural venous sinus, the implementations are not so limited and certain implementations may also be applicable to other uses. For example, the catheters can be adapted for delivery to different neuroanatomies, such as cerebral arteries, subclavian, vertebral, carotid arteries as well as to the coronary anatomy or peripheral vascular anatomy, to name only a few possible applications. Although the systems described herein are described as being useful for treating a particular condition or pathology, that the condition or pathology being treated may vary and are not intended to be limiting.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The catheter system disclosed herein may be packaged together in a single package, where the catheters and catheter advancement element are packaged in a coil tube. The finished package would be sterilized using sterilization methods such as Ethylene oxide or radiation and labeled and boxed. Instructions for use may also be provided in-box or through an internet link printed on the label.

What is claimed is:

1. A method of performing a medical procedure at a treatment site in a dural venous sinus of a brain of a patient, the method comprising:
positioning a system of devices into an advancement configuration, the system of devices comprising:
a catheter having a catheter lumen, an inner diameter and a distal end; and
an inner member sized and shaped to slide within the catheter lumen, wherein the inner member defines a single lumen and has a distal portion, the distal portion having a first outer diameter that tapers distally to a second outer diameter that is smaller than the first outer diameter, wherein the inner member transitions in flexibility from a proximal end of the inner member to a distal end of the inner member, the distal end of the inner member being more flexible than the distal end of the catheter, and
wherein, when positioned in the advancement configuration, the inner member extends coaxially through the catheter lumen until the distal portion of the inner member is positioned distal to the distal end of the catheter;
advancing the catheter and the flexible inner member retrograde to a target location distal to a sigmoid sinus relative to an access point of entry while the system of devices is positioned in the advancement configuration;
positioning the catheter at the treatment site in the dural venous sinus, the treatment site comprising an occlusion;
removing the inner member from the patient; and
treating the occlusion through the catheter to restore outflow of blood from the brain through the dural venous sinus.

2. The method of claim 1, wherein the step of treating comprises removing thrombotic material in the dural venous sinus by applying aspiration to the catheter.

3. The method of claim 1, wherein the step of treating comprises stenting the occlusion in the dural venous sinus with a stent delivered through the catheter.

4. The method of claim 1, wherein the inner member further comprises a proximal segment, wherein the proximal segment comprises a hypotube.

5. The method of claim 1, wherein the inner member further comprises an intermediate segment comprising an unreinforced polymer having a first durometer and the distal portion comprises a polymer different from the unreinforced polymer of the intermediate segment.

6. The method of claim 5, wherein the polymer of the distal portion has a second durometer less than the first durometer.

7. The method of claim 1, wherein the distal portion has a tapered portion that tapers distally from the first outer diameter of between 0.048″ and 0.080″ to the second outer diameter of about 0.031″ up to about 0.048″ over a length between 0.5 cm and 4.0 cm.

8. The method of claim 1, wherein the inner member has a length configured to extend from outside a patient's body, through a femoral artery, and beyond the sigmoid sinus.

9. The method of claim 1, wherein the inner member tapered portion has a taper angle of a wall of the tapered portion relative to a center line of the tapered portion is between 0.9 to 1.6 degrees.

10. The method of claim 9, wherein the inner member tapered portion has a flexibility, shape, and taper length configured to be atraumatically delivered to a venous sinus.

11. The method of claim 1, wherein the first outer diameter of the inner member and the inner diameter of the catheter lumen have a close fit configured to allow the catheter and the inner member to be advanced in the advancement configuration through tortuous, branched venous anatomy to reach the target location for treatment in the distal venous sinuses and aids in preventing the distal end of the catheter from catching on branches and tributaries.

12. The method of claim 1, wherein the second outer diameter is about ½ of the first outer diameter, about 40% of the first outer diameter, or about 65% of the first outer diameter.

13. The method of claim 1, wherein the second outer diameter is at a distal-most terminus of the inner member.

14. The method of claim 1, wherein the single lumen of the flexible elongate body has an inner diameter of less than 0.024 inches.

15. The method of claim 1, further comprising extending a guidewire within the single lumen so that a distal-most end of the guidewire is housed within the inner member proximal to a distal opening from the single lumen.

16. The method of claim 1, wherein the lengths of the catheter and the inner member are configured to extend to the venous sinus and the flexibility of the catheter advancement device is sufficient to reach the venous sinus.

17. A method for performing a medical procedure at a treatment site in a dural venous sinus of a brain of a patient, the method comprising:
advancing a catheter and a flexible inner member retrograde to a target location distal to a sigmoid sinus relative to an access point of entry;
positioning the catheter at the treatment site in the dural venous sinus;
removing the inner member from the patient; and
advancing a treatment device through the catheter to the treatment site.

18. The method of claim 17, wherein the treatment device comprises an aspiration device, an implant delivery device, or a cerebral treatment device.

19. A method of performing a medical procedure at a treatment site in a dural venous sinus of a brain of a patient, the method comprising:
advancing a system of devices retrograde to a target location distal to a sigmoid sinus relative to an access point of entry, the system of devices comprising:
a catheter having a catheter lumen, an inner diameter and a distal end; and
an inner member sized and shaped to slide within the catheter lumen, wherein the inner member has a distal portion having a first outer diameter that tapers distally to a second outer diameter that is smaller than the first outer diameter, and wherein, when positioned in the advancement configuration, the inner member extends coaxially through the catheter lumen and the distal portion of the inner member is positioned distal to the distal end of the catheter;
positioning the catheter at the treatment site in the dural venous sinus, the treatment site comprising an occlusion; and
treating the occlusion with an expandable implant to restore outflow of blood from the brain through the dural venous sinus.

20. The method of claim 19, wherein the expandable implant is a stent deployable from a delivery system from a compressed configuration to a first expanded configuration having a first cross-sectional shape.

21. The method of claim 20, wherein the stent is designed to be formed from the first expanded configuration having the first cross-sectional shape into a second expanded configuration having a second cross-sectional shape.

22. The method of claim 21, wherein the second cross-sectional shape is different from the first cross-sectional shape.

23. The method of claim 22, wherein the first cross-sectional shape is rounded and the second cross-sectional shape is non-round.

24. The method of claim 23, wherein the non-round second cross-sectional shape is a multi-sided shape having a plurality of rounded corners and a plurality of substantially flat sides.

25. The method of claim 23, wherein the non-round second cross-sectional shape is oval, square, or triangular.

26. The method of claim 21, wherein the second cross-sectional shape is a distortion of the first cross-sectional shape.

27. The method of claim 21, wherein the second cross-sectional shape is larger in diameter than the first cross-sectional shape.

28. An implantable device comprising:
a tubular member defining a lumen having a longitudinal axis from a proximal end to a distal end of the tubular member, the tubular member configured to transition from an unexpanded configuration towards a fully expanded, deployed configuration,
wherein the unexpanded configuration has a first cross-sectional shape taken perpendicular to the longitudinal axis that is substantially round without sides and the deployed configuration has a second cross-sectional shape taken perpendicular to the longitudinal axis that is substantially non-round and multi-sided.

29. The device of claim 28, wherein the first cross-sectional shape is circular, oval, or elliptical.

30. The device of claim 28, wherein the second cross-sectional shape is square or triangular.

31. The device of claim 30, wherein the second cross-sectional shape has rounded corners and generally flat sides.

32. The device of claim 31, wherein the rounded corners have a tighter bend radius upon expansion of the tubular member and are formed by thinner, more malleable struts than struts forming the generally flat sides formed by thicker, less malleable struts.

33. The device of claim 28, wherein the tubular member is further configured to transition from the unexpanded configuration to a transitional configuration before transitioning into the deployed configuration.

34. The device of claim 33, wherein the tubular member is configured to be reoriented relative to the anatomy when in the transitional configuration and before transitioning to the deployed configuration.

35. The device of claim 33, wherein the transitional configuration has a transitional cross-sectional shape taken perpendicular to the longitudinal axis that is substantially round without sides, the transitional cross-sectional shape being the same shape or a distorted version of the first cross-sectional shape and having a transitional diameter that is larger than a diameter across the first cross-sectional shape.

36. The device of claim 28, wherein the second cross-sectional shape is configured to mate with luminal dimensions of a venous sinus.

37. A system for implantation of a device in a venous sinus luminal space, the system comprising:
a delivery system comprising:
an inner member; and
an outer member;
a tubular member formed by a plurality of struts that define a lumen having a longitudinal axis from a proximal end to a distal end of the tubular member, the tubular member mounted on the inner member and constrained by the outer member during delivery, wherein the tubular member is configured to transition from an unexpanded configuration towards a fully expanded, deployed configuration, wherein the unexpanded configuration has a first cross-sectional shape taken perpendicular to the longitudinal axis that is substantially round without sides and the deployed configuration has a second cross-sectional shape taken perpendicular to the longitudinal axis that is substantially non-round and multi-sided; and
an outer catheter configured to receive the tubular member constrained by the outer member in the unexpanded configuration for delivery.

38. The system of claim 37, wherein the inner member comprises a recessed area located a distance away from a distal-most end of the inner member, the recessed area having an outer diameter that is less than an outer diameter of the inner member proximal to the recessed area.

39. The system of claim 38, wherein the outer member is configured to be retracted and the tubular member is self-expandable upon retraction of the outer tubular member towards the deployed configuration.

40. The system of claim 38, wherein the recessed area comprises at least one radiopaque marker configured to identify a location of the tubular member within the recessed area.

41. The system of claim 37, wherein the inner member comprises an atraumatic distal end region.

42. The system of claim 37, wherein the first cross-sectional shape is circular, oval, or elliptical.

43. The system of claim 37, wherein the second cross-sectional shape is square or triangular.

44. The system of claim 43, wherein the second cross-sectional shape has rounded corners and generally flat sides.

45. The system of claim 44, wherein the rounded corners have a tighter bend radius upon expansion of the tubular member and are formed by thinner, more malleable struts than struts forming the generally flat sides formed by thicker, less malleable struts.

46. The system of claim 37, wherein the tubular member is further configured to transition from the unexpanded configuration to a transitional configuration before transitioning into the deployed configuration.

47. The system of claim 46, wherein the transitional configuration has a transitional cross-sectional shape taken perpendicular to the longitudinal axis that is substantially round without sides, the transitional cross-sectional shape being the same shape or a distorted version of the first cross-sectional shape and having a transitional diameter that is larger than a diameter across the first cross-sectional shape.

48. The system of claim 37, wherein the second cross-sectional shape is configured to mate with luminal dimensions of a venous sinus.
